(19) United States
(12) Patent Application Publication
(10) Pub. No.: US 2006/0134650 A1
(43) Pub. Date: Jun. 22, 2006

(54) Methylation-sensitive restriction enzyme endonuclease method of whole genome methylation analysis

(75) Inventor: Kevin Gunderson, Encinitas, CA (US)

Correspondence Address: ILLUMINA, INC., 9885 TOWNE CENTRE DRIVE, SAN DIEGO, CA, 92121-1975, US

(73) Assignee: Illumina, Inc., San Diego, CA (US)

(21) Appl. No.: 11/019,652

(22) Filed: Dec. 21, 2004

(51) Int. Cl. C12Q 1/68; C12P 19/34

(52) U.S. Cl. 435/6; 435/91.2

(57) ABSTRACT

The invention provides methods of identifying a plurality of reactive recognition sites for a restriction endonuclease in genomic DNA. In particular embodiments, the methods can be used to identify methylation state of a plurality of CpG target sites in genomic DNA. The method can include steps of treating genomic DNA with a restriction endonuclease, thereby producing genomic DNA fragments; ligating the fragments, thereby forming a concatenated DNA; and identifying sequence portions of the concatenated DNA that are re-ordered compared to the genomic DNA.

This invention was made with government support under grant number 1 R43 CA103406-01 awarded by the National Cancer Institute. The United States Government has certain rights in this invention.

### BACKGROUND OF THE INVENTION

This invention relates generally to detection of nucleic acid modifications, and more specifically to detection of DNA methylation.

DNA methylation is widespread and plays an important role in the regulation of gene expression in development, differentiation, and several conditions or diseases such as multiple sclerosis, diabetes, schizophrenia, aging, and cancer. Methylation in particular gene regions, such as promoter regions, can inhibit the expression of these genes. This gene silencing effect is believed to be accomplished through the interaction of methylcytosine binding proteins with other structural components of chromatin resulting in histone deacetylation and chromatin structure changes that inhibit interaction of transcription factors with the DNA.

Genomic imprinting in which imprinted genes are preferentially expressed from either the maternal or paternal allele involves DNA methylation. In vertebrates, the DNA methylation pattern is established early in embryonic development and, in general, the distribution of 5-methylcytosine along the chromosome is maintained during the life span of the organism. Stable transcriptional silencing facilitates normal development, and is associated with several epigenetic modifications. If methylation patterns are not properly established or maintained, various disorders like mental retardation, immune deficiency and sporadic or inherited cancers can result.

Changes in DNA methylation have been recognized as one of the most common molecular alterations in human neoplasia. Hypermethylation of CpG islands located in the promoter regions of tumor suppressor genes are the most frequent mechanisms for gene inactivation in cancers. In contrast, a global hypomethylation of genomic DNA and loss of IGF imprinting are observed in tumor cells; and a correlation between hypomethylation and increased gene expression has been reported for many oncogenes. In addition, monitoring global changes in methylation pattern has been applied to molecular classification of cancers. Furthermore, gene hypermethylation has been associated with clinical risk groups in neuroblastoma and response to tamoxifen in breast cancer.

Methylation is also important for the initiation and the maintenance of an inactive X-chromosome in females, a mechanism important for balancing expression from the two copies of each X-chromosome present in the female genome. Rett syndrome is an X-linked dominant disease that results from improper inactivation of an X-Chromosome. This in turn is caused by mutation of MeCP2 gene, which normally represses transcription by binding methylated CpG residues and mediating chromatin remodeling.

A major goal of epidemiological genetics is to relate specific conditions and diseases to the genotypes of specific genes or to the potential differential expression levels of each allele of the genes. DNA methylation data can provide valuable information regarding gene expression. It should be feasible to use methylation patterns to classify and predict different disease states such as different types of cancer, different stages of cancer, different outcomes for cancer therapeutics or patient survival. However, methylation patterns can be complex involving several different genes and in many cases several different sites within each gene.

Thus, the discovery or identification of associations will be benefited by the ability to conveniently evaluate methylation at several genetic locations in an individual's genome. Further benefits will be provided by the ability to efficiently evaluate methylation patterns in the genomes of large numbers of individuals, for example, in a multiplexed assay. The present invention provides these advantages and others as well.

### BRIEF SUMMARY OF THE INVENTION

The invention provides a method of identifying a plurality of reactive recognition sites for a restriction endonuclease in genomic DNA. The method includes the steps of (a) providing an isolated native genomic DNA having a first sequence, wherein the genomic DNA has a plurality of reactive recognition sites for a restriction endonuclease; (b) cleaving the genomic DNA at the recognition sites with the restriction endonuclease, thereby producing genomic DNA fragments having portions of the first sequence; (c) ligating the fragments, thereby forming a concatenated DNA having the portions in a second sequence, wherein the portions are re-ordered in the second sequence compared to the first sequence; and (d) identifying a plurality of the portions that are re-ordered, thereby identifying a plurality of reactive recognition sites for the restriction endonuclease in the genomic DNA.

The invention further provides a method of identifying a reactivity state of a plurality of recognition sites for a restriction endonuclease in genomic DNA. The method includes the steps of (a) providing an isolated native genomic DNA having a first sequence, wherein the genomic DNA has a plurality of recognition sites for a restriction endonuclease, the recognition sites having recognition sequences in the first sequence, wherein the recognition sites have a first reactivity state or second reactivity state, wherein the genomic DNA has portions of the first sequence that are adjacent to the recognition sequences; (b) cleaving the genomic DNA at the recognition sites with the restriction endonuclease, thereby producing genomic DNA fragments having the portions of the first sequence; (c) ligating the fragments, thereby forming a concatenated DNA having a second sequence, wherein the portions are re-ordered in the second sequence compared to the first sequence if the recognition sites are in the first reactivity state and wherein the portions are ordered the same in the second sequence compared to the first sequence if the recognition sites are in the second reactivity state; and (d) identifying the order for a plurality of the portions, thereby identifying the reactivity state of the plurality of recognition sites for the restriction endonuclease in the genomic DNA.

Also provides is a method of identifying methylation state of a plurality of CpG target sites in genomic DNA. The method includes the steps of (a) providing an isolated native genomic DNA having a first sequence, wherein the genomic DNA has CpG target sites at a plurality of different locations, wherein the CpG target sites have a cytosine having a 5-methyl moiety or a 5-hydrogen moiety, wherein the genomic DNA has portions that are adjacent to the CpG target sites; (b) cleaving the genomic DNA at the CpG target sites that have the 5-methyl cytosine, thereby producing genomic DNA fragments having the portions; (c) ligating the fragments, thereby forming a concatenated DNA having the portions in a second sequence, wherein the portions are re-ordered in the second sequence compared to the first sequence if the CpG target sites have cytosine having the 5-methyl moiety and wherein the portions are ordered the same in the second sequence compared to the first sequence if the CpG target sites have cytosine having the 5-hydrogen moiety; and (d) identifying the order for a plurality of the portions, thereby identifying methylation state of the plurality of CpG target sites in the genomic DNA.

### BRIEF DESCRIPTION OF THE DRAWINGS

FIG. 1 shows an exemplary method for identifying methylated and nonmethylated restriction sites in a genomic DNA. Different sequence portions of genomic DNA are represented by bars having different fill patterns separated by methylated restriction sites (C^{m}) or unmethylated restriction sites (C). Solid phase-attached, capture probes that complement particular sequence portions of genomic DNA are shown as bars attached to black circles having similar pattern to the sequence portions of the genomic DNA. Labeled nucleotides added to capture probes in the presence of a complementary target are shown as asterisks.

### DEFINITIONS

As used herein, the term “restriction endonuclease” or “RE” is intended to mean an agent that recognizes a specific nucleotide sequence in a nucleic acid and cleaves the nucleic acid. A restriction endonuclease can recognize a sequence that is, for example, 4, 5, 6, 7 or more nucleotides long. A restriction endonuclease can recognize more than one sequence, for example, two or more variants of a degenerate sequence that includes one of two or more different nucleotides at a particular position. For example, the Hinc II restriction endonuclease recognizes the degenerate sequence GTPyPuAC which represents the four sequences GTCGAC, GTCAAC, GTTGAC and GTTAAC. Alternatively, a restriction endonuclease can be specific for a single recognition sequence.

As used herein, the term “recognition site,” when used in reference to a nucleic acid, is intended to mean a portion of the nucleic acid having a nucleotide sequence that specifically binds to a particular binding moiety such as a restriction endonuclease. Typically, a restriction endonuclease recognition site is cleaved by a restriction endonuclease. In some embodiments, the portion of a nucleic acid that is cleaved by a restriction endonuclease can be adjacent to or removed from the recognition site of the restriction endonuclease.

As used herein, the term “reactive recognition site,” when used in reference to a nucleic acid, is intended to mean a portion of a nucleic acid that specifically binds to a particular binding moiety such that the nucleic acid is covalently modified. For example, a reactive RE recognition site is a portion of a nucleic acid that specifically binds to a particular restriction endonuclease such that the nucleic acid is cleaved by the restriction endonuclease. Whether or not a recognition site is reactive will be understood in terms of its interaction (or lack of interaction) with a particular enzyme or agent. In this regard, a recognition site can have substantially reduced or no reactivity for a particular methylation sensitive restriction endonuclease (MSRE) when it has a methylated base and the same site can be reactive for the MSRE when it lacks the methyl moiety on the base. For example, the CCCGGG recognition site is a reactive recognition site for the SmaI restriction endonuclease, being cleaved when the third cytosine is unmethylated, but cleavage of the site by SmaI is inhibited when the third cytosine is methylated. However, the CCCGGG recognition site is reactive for the XmaI restriction endonuclease, being cleaved, when the third C is methylated. SmaI and XmaI are isoschizomers due to the ability to recognize the same base sequence. In a further example, MboI and Sau3AI are isoschizomers that recognize and cleave the sequence GATC. Under typical conditions, MboI does not cleave GmATC, while digestion of GATC by Sau3AI is unaffected by methylation.

Alternatively, a reactive recognition site can have substantially reduced or no reactivity for a particular methylation sensitive restriction endonuclease (MSRE) when it lacks a methyl moiety on a particular base and the same site can be inactive for the MSRE when the base is methylated. For example, McrBC (New England BioLabs) cleaves methylated DNA having the consensus site Pu^{m}C(N40-3000) Pu^{m}C, where Pu is a purine, such as adenine or guanine, and the superscript m refers to presence of a methyl moiety. A further example is PvuRtslI which cleaves DNA containing 5-hydroxymethyl cytosine, but is inactive toward non-methylated DNA.

As used herein, the term “methylation sensitive restriction endonuclease” or “MSRE” is intended to mean an agent that recognizes a specific nucleotide sequence in a nucleic acid and cleaves the nucleic acid, wherein presence of a methylated base in the specific nucleotide sequence increases or decreases the cleavage activity compared to absence of the methylated base. Exemplary MSREs include, but are not limited to NotI, SmaI, XmaI, MboI, BstBI, ClaI, MluI, NaeI, NarI, PvuI, SacII, SalI, HpaII, and HhaI. Other useful MSREs are described, for example, in McClelland et al., Nucl. Acids Res. 22:3640-3659 (1994) or in technical materials available from commercial vendors such as New England Biolabs (Beverly, Mass.), Promega (Madison, Wis.), or Invitrogen (Carlsbad, Calif.).

Specific binding between two binding partners, such as a nucleic acid and a restriction enzyme, is understood to mean preferential binding of one partner to another compared to binding of the partner to other components or contaminants in the system. Depending upon the particular binding conditions used, the dissociation constants of the pair can be, for example, less than about 10⁻⁴, 10⁻⁵, 10⁻⁶, 10⁻⁷, 10⁻⁸, 10⁻⁹ 10⁻¹⁰, 10⁻¹¹, or 10⁻¹² M⁻¹.

As used herein, the term “genomic DNA” or “gDNA” is intended to mean one or more chromosomal polymeric deoxyribonucleotide molecules occurring naturally in the nucleus of a eukaryotic cell or in a prokaryote, virus, mitochondrion or chloroplast and containing sequences that are naturally transcribed into RNA A gDNA can also include sequences that are not naturally transcribed into RNA by the cell. A chromosome of a eukaryotic cell contains at least one centromere, two telomeres, one origin of replication, and one sequence that is not transcribed into RNA by the eukaryotic cell including, for example, an intron or transcription promoter. A chromosome of a prokaryotic cell contains at least one origin of replication and one sequence that is not transcribed into RNA by the prokaryotic cell including, for example, a transcription promoter. A eukaryotic gDNA can be distinguished from prokaryotic, viral or organellar gDNA, for example, according to the presence of introns in eukaryotic gDNA and absence of introns in the gDNA of the others. A gDNA used in the invention can include all or part of an organism's genome.

As used herein, the term “sequence,” when used in reference to a nucleic acid molecule, is intended to mean a linear series of bases in the nucleic acid molecule.

As used herein, the term “isolated,” when used in reference to a biological substance, is intended to mean removed from at least a portion of the molecules associated with the substance in its native environment or occurring with the substance in its native environment. Accordingly, the term “isolating,” when used in reference to a biological substance, is intended to mean removing the substance from its native environment or removing at least a portion of the molecules associated with or occurring with the nucleic acid or substance in its native environment. Exemplary substances that can be isolated include, without limitation, nucleic acids, proteins, chromosomes, cells, tissues or the like. An isolated biological substance, such as a nucleic acid, can be essentially free of other biological substances. An isolated nucleic acid can be at least about 90%, 95%, 99% or 100% free of non-nucleotide material naturally associated with it. An isolated nucleic acid can, for example, be essentially free of other nucleic acids such that its sequence is increased to a significantly higher fraction of the total nucleic acid present in the solution of interest than in the cells from which the nucleic acid was taken. For example, an isolated nucleic acid can be present at a 2, 5, 10, 50, 100 or 1000 fold or higher level than other nucleic acids in vitro relative to the levels in the cells from which it was taken. This can be caused by preferential reduction in the amount of other DNA or RNA present, or by a preferential increase in the amount of the specific DNA or RNA sequence, or by a combination of the two.

As used herein, the term “cleaving,” when used in reference to a nucleic acid, is intended to mean breaking a covalent bond that joins two nucleotide subunits of the nucleic acid, thereby producing two separate fragments of the nucleic acid. A nucleic acid can be cleaved, for example, by breaking a phosphodiester bond between two nucleotides to produce a first fragment having a 3′ hydroxyl and a second fragment having a 5′ phosphate. Other exemplary bonds that can be broken in accordance with the definition include, but are not limited to, one or more bonds in a ribose, deoxyribose or other sugar moiety of a nucleic acid. Any of a variety of bonds in the backbone of a nucleic acid can be broken resulting in cleavage including, for example, bonds that replace phosphodiesters and/or ribose sugars in analog structures such as those set forth in further detail below.

As used herein, the term “ligating,” when used in reference to two nucleic acid molecules, is intended to mean covalently attaching the two nucleic acid molecules to form a single molecule having a single linear sequence of nucleotides. For example, a first nucleic acid having a terminal 3′ hydroxyl can be covalently attached to a second nucleic acid having a terminal 5′ phosphate by formation of a phosphodiester bond between the 3′ hydroxyl and 5′ phosphate. Such covalent attachment can be catalyzed by an enzyme such as a ligase or be chemicals that are reactive with nucleic acids.

As used herein, the term “concatenated,” when used in reference to a nucleic acid having two or more portions, is intended to mean having the portions present in a linear series or sequence.

As used herein, the term “re-ordered” is intended to mean arranged in a different way. For example, a first sequence having two contiguous portions is re-ordered compared to a second sequence having the two portions no longer contiguous. Accordingly, the two portions have different contiguity in the first and second sequences. Furthermore, two or more sequence portions can occur in a first nucleic acid and in a second nucleic acid such that the relative order of the portions in the first nucleic acid is different from the order of the portions in the second nucleic acid.

As used herein, the term “amplify,” when used in reference to a nucleic acid template, is intended to mean producing one or more copies of the nucleic acid template, or a portion thereof. The copy can be a single stranded or double stranded nucleic acid and can be produced from a single stranded or double stranded template nucleic acid. For example, the copy can be a single stranded nucleic acid copy of one of the strands (or portion thereof) of a double stranded nucleic acid template.

As used herein, the term “reactivity state” is intended to mean one of two or more discrete configurations, attributes, or conditions, wherein each discrete configuration, attribute, or condition provides a different type of function or level of function. Examples of reactivity states include, but are not limited to, presence or absence of a moiety on a nucleic acid such as a methyl moiety on a nucleotide base, or presence or absence of a particular base at a location in a sequence. Exemplary functions include, without limitation, binding affinity or chemical reactivity. More specifically, an exemplary function can be binding affinity for a restriction endonuclease or cleavage reactivity with a restriction endonuclease. Different types of a function that can characterize a reactivity state of a recognition site include, for example, a recognition site in a first state having binding affinity for a first restriction endonuclease versus the recognition site when in a second state having binding affinity for a different restriction endonuclease. Different levels of a function can include, for example, higher binding affinity of a first state for a restriction endonuclease and lower binding affinity of a second state for the same restriction endonuclease.

As used herein, the term “CpG target site” is intended to mean a location in a sequence having a cytosine that is bonded via its 3′ oxygen to the 5′ phosphate of a guanine. The term is intended to be consistent with its meaning in the biological arts. A particular CpG target site can be identified according to its location in a reference sequence such as a genome sequence. Accordingly, a CpG target site in a nucleic acid molecule can be identified according to a sequence flanking the CpG in the molecule and, if desired, comparison to a reference sequence. A sequence useful for identifying a CpG target site can be at least about 5, 7, 10, 12, 15, 18, 20 or more nucleotides in length up to and including a length that allows location of the CpG in a reference sequence. A CpG site can either include a methyl moiety at the cytosine (C) or can lack a methyl moiety at the C.

### DETAILED DESCRIPTION OF PARTICULAR EMBODIMENTS

The invention provides a method of identifying a plurality of reactive recognition sites for a restriction endonuclease in genomic DNA. The method includes the steps of (a) providing an isolated native genomic DNA having a first sequence, wherein the genomic DNA has a plurality of reactive recognition sites for a restriction endonuclease; (b) cleaving the genomic DNA at the recognition sites with the restriction endonuclease, thereby producing genomic DNA fragments having portions of the first sequence; (c) ligating the fragments, thereby forming a concatenated DNA having the portions in a second sequence, wherein the portions are re-ordered in the second sequence compared to the first sequence; and (d) identifying a plurality of the portions that are re-ordered, thereby identifying a plurality of reactive recognition sites for the restriction endonuclease in the genomic DNA.

The reactivity of a recognition site for a restriction endonuclease (RE) can be influenced by any of several factors such as the sequence of nucleotides at the site, the sequence of nucleotides adjacent to or proximal to the site, the conformational structure of the nucleic acid at or near the recognition site, the presence or absence of a covalent modifications to bases at or near the recognition site, or the presence or absence of a binding agent, such as a nucleic acid binding protein, that binds at or near the recognition site. A method of the invention can be used to determine the reactivity of the recognition sites and in turn identify the presence or absence of factors such as those set forth above.

For example, a method of the invention can be used to determine presence or absence of a methylated base at a recognition site. A recognition site can be methylated at one or more of several different locations that influence reactivity of the recognition site for a particular RE such as, 5-methyl cytosine (5mC), N⁶-methyl adenine (6mA), 5-hydroxymethyluracil (5hmU), N⁷-methyl guanine (7mG), or N⁴-methyl cytosine (4mC).

Reactive recognition sites for one or more RE can be identified using an RE that has differential restriction activity for the recognition site in a first form when compared to another form. For example, methylated bases, and their sequence location, can be identified in a gDNA using a methylation sensitive restriction endonuclease (MSRE) that is inhibited from cleaving a methylated recognition site in the gDNA, but is capable of cleaving the recognition site when the site lacks the inhibitory methyl moiety. Thus, a useful method can utilize a restriction endonuclease that is methylation sensitive wherein the reactive restriction sites are unmethylated, thereby allowing identification of a plurality of unmethylated recognition sites for the restriction endonuclease in the gDNA.

Similarly, a nucleic acid binding protein bound to a nucleic acid can be detected and the sequence to which the protein is bound identified. For example, if the nucleic acid binding protein binds to a restriction site in a nucleic acid, thereby preventing the site from being cleaved by its RE, then the presence of the protein at the site can be identified due to inhibition of the RE. Accordingly, the method can be used to map the binding sites of a DNA binding protein via the use of an appropriate restriction enzyme or nuclease.

An advantage provided by the invention is the ability to identify a plurality of reactive recognition sites for an RE in gDNA. The methods are ideally suited to the analysis of large genomes such as those typically found in eukaryotic unicellular and multicellular organisms. Exemplary eukaryotic gDNA that can be used in a method of the invention includes, without limitation, that from a mammal such as a rodent, mouse, rat, rabbit, guinea pig, ungulate, horse, sheep, pig, goat, cow, cat, dog, primate, human or non-human primate; a plant such as Arabidopsis thaliana, corn (Zea mays), sorghum, oat (oryza sativa), wheat, rice, canola, or soybean; an algae such as Chlamydomonas reinhardtii; a nematode such as Caenorhabditis elegans; an insect such as Drosophila melanogaster, mosquito, fruit fly, honey bee or spider; a fish such as zebrafish (Danio rerio); a reptile; an amphibian such as a frog or Xenopus laevis; a dictyostelium discoideum; a fungi such as pneumocystis carinii, Takifugu rubripes, yeast, Saccharamoyces cerevisiae or Schizosaccharomyces pombe; or a plasmodium falciparum. A method of the invention can also be used to analyze RE recognition sites of smaller genomes such as those from a prokaryote such as a bacterium, Escherichia coli, staphylococci or mycoplasma pneumoniae; an archae; a virus such as Hepatitis C virus or human immunodeficiency virus; or a viroid.

A gDNA used in the invention can have one or more chromosomes. For example, a prokaryotic gDNA having one chromosome can be used. Alternatively, a eukaryotic gDNA including a plurality of chromosomes can be used in a method of the invention. Thus, the methods can be used, for example, analyze a gDNA having n equal to at least 2, 4, 6, 8, 10, 15, 20, 23, 25, 30, or 35 or more chromosomes, where n is the haploid chromosome number and the diploid chromosome count is 2n. The size of a gDNA used in a method of the invention can also be measured according to the number of base pairs or nucleotide length of the chromosome complement. Exemplary size estimates for some of the genomes that are useful in the invention are about 3.1 Gb (human), 2.7 Gb (mouse), 2.8 Gb (rat), 1.7 Gb (zebrafish), 165 Mb (fruitfly), 13.5 Mb (S. cerevisiae), 390 Mb (fugu), 278 Mb (mosquito) or 103 Mb (C. elegans). Those skilled in the art will recognize that genomes having sizes other than those exemplified above including, for example, smaller or larger genomes, can be used in a method of the invention.

A gDNA used in a method of the invention can be all or part of the gDNA from an individual organism. Accordingly, a genomic DNA used in the methods can have a sequence that is at least about 10 kb, 20 kb, 30 kb, 40 kb, 50 kb, 100 kb, 500 kb, 1 mb, 10 mb or 50 mb in length up to and including the length of a full genome such as those exemplified above.

Genomic DNA can be isolated from one or more cells, bodily fluids or tissues. Known methods can be used to obtain a bodily fluid such as blood, sweat, tears, lymph, urine, saliva, semen, cerebrospinal fluid, feces or amniotic fluid. Similarly, known biopsy methods can be used to obtain cells or tissues such as buccal swab, mouthwash, surgical removal, biopsy aspiration or the like. Genomic DNA can also be obtained from one or more cell or tissue in primary culture, in a propagated cell line, a fixed archival sample, forensic sample or archeological sample.

Exemplary cell types from which gDNA can be obtained in a method of the invention include, without limitation, a blood cell such as a B lymphocyte, T lymphocyte, leukocyte, erythrocyte, macrophage, or neutrophil; a muscle cell such as a skeletal cell, smooth muscle cell or cardiac muscle cell; germ cell such as a sperm or egg; epithelial cell; connective tissue cell such as an adipocyte, fibroblast or osteoblast; neuron; astrocyte; stromal cell; kidney cell; pancreatic cell; liver cell; or keratinocyte. A cell from which gDNA is obtained can be at a particular developmental level including, for example, a hematopoietic stem cell or a cell that arises from a hematopoietic stem cell such as a red blood cell, B lymphocyte, T lymphocyte, natural killer cell, neutrophil, basophil, eosinophil, monocyte, macrophage, or platelet. Other cells include a bone marrow stromal cell (mesenchymal stem cell) or a cell that develops therefrom such as a bone cell (osteocyte), cartilage cells (chondrocyte), fat cell (adipocyte), or other kinds of connective tissue cells such as one found in tendons; neural stem cell or a cell it gives rise to including, for example, a nerve cells (neuron), astrocyte or oligodendrocyte; epithelial stem cell or a cell that arises from an epithelial stem cell such as an absorptive cell, goblet cell, Paneth cell, or enteroendocrine cell; skin stem cell; epidermal stem cell; or follicular stem cell. Generally any type of stem cell can be used including, without limitation, an embryonic stem cell, adult stem cell, totipotent stem cell or pluripotent stem cell.

A cell from which a gDNA sample is obtained for use in the invention can be a normal cell or a cell displaying one or more symptom of a particular disease or condition. Thus, a gDNA used in a method of the invention can be obtained from a cancer cell, neoplastic cell, apoptotic cell, senescent cell, necrotic cell or the like. Those skilled in the art will know or be able to readily determine methods for isolating gDNA from a cell, fluid or tissue using methods known in the art such as those described in Sambrook et al., Molecular Cloning: A Laboratory Manual, 3rd edition, Cold Spring Harbor Laboratory, New York (2001) or in Ausubel et al., Current Protocols in Molecular Biology, John Wiley and Sons, Baltimore, Md. (1998).

A method of the invention can further include steps of isolating a particular type of cell or tissue. Exemplary methods that can be used to isolate a particular cell from other cells in a population include, but are not limited to, Fluorescent Activated Cell Sorting (FACS) as described, for example, in Shapiro, Practical Flow Cytometry, 3rd edition Wiley-Liss; (1995), density gradient centrifugation, or manual separation using micromanipulation methods with microscope assistance. Exemplary cell separation devices that are useful in the invention include, without limitation, a Beckman JE-6 centrifugal elutriation system, Beckman Coulter EPICS ALTRA computer-controlled Flow Cytometer-cell sorter, Modular Flow Cytometer from Cytomation, Inc., Coulter counter and channelyzer system, density gradient apparatus, cytocentrifuge, Beckman J-6 centrifuge, Cytopeia InFlux cell sorter, EPICS V dual laser cell sorter, or EPICS PROFILE flow cytometer. A tissue or population of cells can also be removed by surgical techniques. For example, a tumor or cells from a tumor can be removed from a tissue by surgical methods, or conversely non-cancerous cells can be removed from the vicinity of a tumor.

A gDNA can be prepared for use in a method of the invention by lysing a cell that contains the DNA. Typically, a cell is lysed under conditions that substantially preserve the integrity of the cell's gDNA. In particular, exposure of a cell to alkaline pH can be used to lyse a cell in a method of the invention while causing relatively little damage to gDNA. Any of a variety of basic compounds can be used for lysis including, for example, potassium hydroxide, sodium hydroxide, or the like. Additionally, relatively undamaged gDNA can be obtained from a cell lysed by an enzyme that degrades the cell wall. Cells lacking a cell wall either naturally or due to enzymatic removal can also be lysed by exposure to osmotic stress. Other conditions that can be used to lyse a cell include exposure to detergents, mechanical disruption, sonication heat, pressure differential such as in a French press device, or Dounce homogenization. Agents that stabilize gDNA can be included in a cell lysate or isolated gDNA sample including, for example, nuclease inhibitors, chelating agents, salts buffers and the like. Methods for lysing a cell to obtain gDNA can be carried out under conditions known in the art as described, for example, in Sambrook et al., supra (2001) or in Ausubel et al., supra, (1998).

In particular embodiments of the invention, a crude cell lysate containing gDNA can be directly used without further isolation of the gDNA. Alternatively, a gDNA can be further isolated from other cellular components. Accordingly, a method of the invention can be carried out on purified or partially purified gDNA. Genomic DNA can be isolated using known methods including, for example, liquid phase extraction, precipitation, solid phase extraction, chromatography and the like. Such methods are often referred to as minipreps and are described for example in Sambrook et al., supra, (2001) or in Ausubel et al., supra, (1998) or available from various commercial vendors including, for example, Qiagen (Valencia, Calif.) or Promega (Madison, Wis.).

A gDNA used in a method of the invention will typically include a larger number of reactive restriction sites. For example, an RE that is specific for a 4 nucleotide recognition site will cleave a gDNA, on average every 256 nucleotides. Using a similar estimate, assuming a random distribution of A, G, C, and T in a genome, an RE specific for a 5 nucleotide recognition site will cleave the genome on average every 1024 nucleotides and an RE specific for a 6 nucleotide recognition site will cleave the genome on average every 4096 nucleotides. Accordingly, a gDNA can have a plurality of recognition sites for one or more REs occurring at 100, 1,000, 10,000, 100,000, 1,000,000 or more locations in the gDNA. Furthermore, cleavage of the gDNA with the appropriate RE will yield a large population of fragments. The methods disclosed herein are well suited for handling large populations of gDNA fragments including, but not limited to, populations having at least about 100, 1,000, 10,000, 100,000, 1,000,000 or more different fragments.

Accordingly, a method of the invention can be used to identify RE recognition sites at a number of locations in a genome sequence including, but not limited to, the numbers or ranges set forth above. Other exemplary pluralities of reactive RE recognition sites that can be identified include one or more RE recognition site at least about 2, 5, 10, 25, 50 or 75 different locations in a genome.

A gDNA, or other nucleic acid, can be contacted with an RE under conditions wherein the RE is specific for a reactive form of a recognition site compared to its inactive form. Those skilled in the art will know or be able to determine appropriate conditions according to that which is known in the art regarding restriction endonuclease activity as described for example in Sambrook et al., supra, (2001) or in Ausubel et al., supra, (1998) or in technical materials available from commercial vendors such as New England Biolabs (Beverly, Mass.), Promega (Madison, Wis.), or Invitrogen (Carlsbad, Calif.). Typically, reactions are allowed to proceed to completion such that a cleavage event occurs for substantially all of the reactive recognition sites.

A gDNA can be treated with a single type of RE or multiple types of REs in a method of the invention. In embodiments where a gDNA is treated with multiple types of REs, the gDNA can be contacted with the REs simultaneously or sequentially. A gDNA can be treated with any desired number of different REs including, for example, at least 2, 3, 4, 5, 10 or more different REs. The REs can be isoschizomers, recognizing the same recognition site sequence, or can recognize different recognition site sequences. The isoschizomers can have different reactivity for a site based on presence or absence of a modification such as methylation as set forth in further detail below, for example, in the context of using controls. Furthermore, the REs can produce genomic fragments having the same or different 5′ or 3′ ends.

A gDNA fragment produced in accordance with a method set forth herein can have a blunt end or a 3′ or 5′ overhang, depending upon the activity of the RE used. If desired, a gDNA fragment can be modified to remove an overhang using for example, a single stranded nuclease. Alternatively, an overhang can be ligated to a double stranded adapter nucleic acid such that a blunt end results. Similarly, an adapter nucleic acid can be ligated to a gDNA having a blunt end or an overhang to introduce a new overhang to the end of the fragment. Such changes to the end of a gDNA fragment can be useful for facilitating ligation under a desired condition. For example, it may be desirable to produce fragments having overhangs to increase ligation specificity over a blunt end ligation reaction.

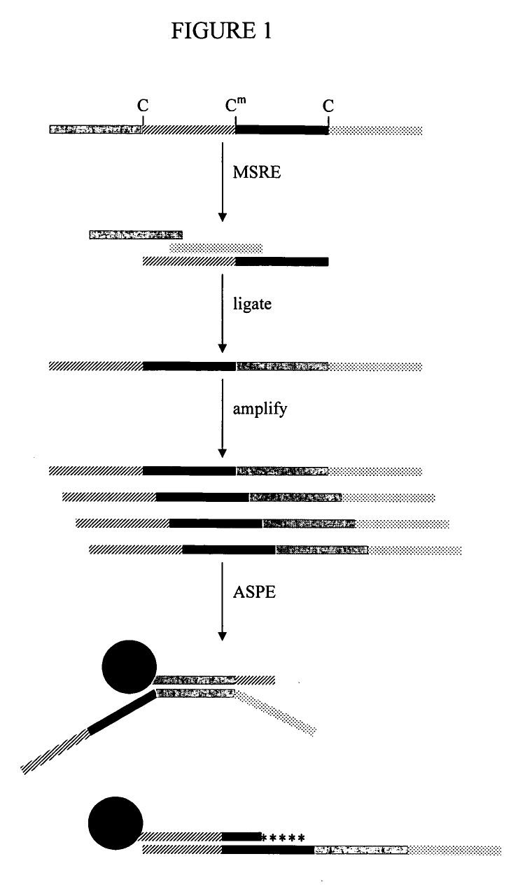

A population of gDNA fragments can be ligated together such that they form a concatenated DNA. For a large gDNA, having RE recognition sequences at several locations, cleavage will produce a correspondingly large population of genome fragments. Each fragment will have a sequence representing a portion of the sequence for the original gDNA. Contacting this population of fragments with a ligation reagent, such as a ligase enzyme, will yield a concatenated DNA product having a sequence that is re-ordered compared to the sequence of the gDNA prior to cleavage. This re-ordering occurs because the gDNA fragments will randomly ligate such that sequence portions that were adjacent in the original gDNA are separated by an intervening sequence corresponding to at least one other portions of the gDNA sequence. Although it is possible for two fragments to ligate such that formerly adjacent portions (in the original gDNA) are also adjacent in the concatenated product, typically the number of gDNA fragments used is sufficiently large that these events would have a low probability of occurrence. Thus, re-ordering of two sequence portions in a concatenated DNA compared to the original gDNA indicates that the RE recognition sequence between the two portions in the original gDNA is reactive. Conversely, absence of re-ordering of two sequence portions generally indicates that the RE recognition sequence between the two portions in the original gDNA is inactive. By way of example, FIG. 1 shows an embodiment in which sequence portions about non-methylated RE recognition sites are re-ordered while sequence portions about methylated RE recognition sites are in the same order as the gDNA that was treated with an MSRE, thereby allowing identification of methylated sites in the gDNA.

A method of the invention can include a step of amplifying a nucleic acid such as a concatenated DNA. An amplified nucleic acid can be produced from a concatenated DNA template such that at least one of the concatenated portions is present in the amplified product. Typically, the amplified product will include several portions and the order of the portions will reflect the order of the portions in the concatenated template. For example, the portions in the amplified product can be in the same order as the concatenated DNA template or in the opposite order, depending if the comparison is made to the template strand or its complement.

A copy of a nucleic acid, such as a concatenated nucleic acid, can be synthesized using random primer amplification. Exemplary techniques for random primer amplification include, without limitation, those based upon PCR, such as PEP-PCR or DOP-PCR. However, amplification in the methods disclosed herein need not include the polymerase chain reaction. Specifically, amplification can be carried out such that sequences are amplified several fold under isothermal conditions. Thus, although an elevated temperature step can be used, for example, to initially denature a nucleic acid template, temperature cycling need not be used. Accordingly, repeated increases in temperature, normally used to denature hybrids during PCR, and repeated return to hybridization temperatures need not be used. Thus, strand-displacement amplification such as random-primer amplification can be used in a method of the invention. Alternative or additional methods that can be used to denature a template nucleic acid include, for example, use of an enzyme such as a helicase, chemical agent such as salt or detergent, pH or the like.

Exemplary isothermal amplification methods that can be used in a method of the invention include, but are not limited to, Multiple Displacement Amplification (MDA) under conditions such as those described in Dean et al., Proc Natl. Acad. Sci USA 99:5261-66 (2002) or isothermal strand displacement nucleic acid amplification as described in U.S. Pat. No. 6,214,587 or 5,043,272. Other non-PCR-based methods that can be used in the invention include, for example, strand displacement amplification (SDA) which is described in Walker et al., Molecular Methods for Virus Detection, Academic Press, Inc., 1995; U.S. Pat. Nos. 5,455,166, and 5,130,238, and Walker et al., Nucl. Acids Res. 20:1691-96 (1992) or hyperbranched strand displacement amplification which is described in Lage et al., Genome Research 13:294-307 (2003). Isothermal amplification methods can be used with the strand-displacing φ29 polymerase or Bst DNA polymerase large fragment, 5′->3′ exo⁻ for random primer amplification. The use of these polymerases takes advantage of their high processivity and strand displacing activity. High processivity allows the polymerases to produce fragments that are 10-20 kb in length. As set forth above, smaller products can be produced under isothermal conditions using polymerases having low processivity and strand-displacing activity.

In particular embodiments, a concatenated nucleic acid or other template can be amplified by a method that utilizes random or degenerate oligonucleotide primed polymerase chain reaction (PCR). An exemplary method is known as primer extension preamplification (PEP). This technique uses random primers in combination with a thermostable DNA polymerase to replicate copies throughout the genome. Exemplary conditions that can be used for PEP-PCR are described in Zhang et al., Proc. Natl. Acad. Sci. USA, 89:5847-51 (1992); Casas et al., Biotechniques 20:219-25 (1996); Snabes et al., Proc. Natl. Acad. Sci. USA, 91:6181-85 (1994,); or Barrett et al., Nucleic Acids Res., 23:3488-92 (1995).

Another amplification method that is useful in the invention is Tagged PCR which uses a population of two-domain primers having a constant 5′ region followed by a random 3′ region as described, for example, in Grothues et al. Nucleic Acids Res. 21(5):1321-2 (1993). The first rounds of amplification are carried out to allow a multitude of initiations on heat denatured template based on individual hybridization from the randomly-synthesized 3′ region. Due to the nature of the 3′ region, the sites of initiation will be random throughout the genome. Thereafter, the unbound primers can be removed and further replication can take place using primers complementary to the constant 5′ region.

A further useful amplification method is degenerate oligonucleotide primed polymerase chain reaction (DOP-PCR) which can be carried out under known conditions such as those described in Cheung et al., Proc. Natl. Acad. Sci. USA, 93:14676-79 (1996) or U.S. Pat. No. 5,043,272. Furthermore modified versions of DOP-PCR, such as those described by Kittler et al. in a protocol known as LL-DOP-PCR (Long products from Low DNA quantities-DOP-PCR) can be used to amplify a template nucleic acid in accordance with the invention (Kittler et al., Anal. Biochem. 300:237-44 (2002)).

Arbitrary-primer PCR can also be used to amplify a template nucleic acid in a method of the invention. Arbitrary-primer PCR can be carried out by replicating a template with a primer under non-stringent conditions such that the primer arbitrarily anneals to various locations in the template. Subsequent PCR steps can be carried out at higher stringency to amplify the fragments generated due to arbitrary priming in the previous step. The length, sequence or both of an arbitrary-primer can be selected in accordance with the probability of priming at particular intervals along the gDNA. In this regard, as primer length increases, the average interval between arbitrarily primed locations will increase, assuming no change in other amplification conditions. Similarly, a primer having a sequence complementary to or similar to a repeated sequence will prime more often, yielding shorter intervals between amplified fragments than a primer that lacks complementarity to the repeated sequence. Arbitrary-primer amplification can be carried out under conditions similar to those described, for example, in Bassam et al., Australas Biotechnol. 4:232-6 (1994). In accordance with the invention, amplification can be carried out under isothermal conditions using an arbitrary primer, low stringency annealing conditions, and a strand-displacing polymerase.

Another method that can be used to amplify a nucleic acid template having a genome sequence is inter-Alu PCR. In this method, primers are designed to anneal to Alu sequences which are repeated throughout the genome. PCR amplification with these primers will yield fragments flanked by Alu repeats. Those skilled in the art will recognize that similar methods can be carried out with primers that anneal to other repeated sequences in a genome of interest such as transcription regulatory regions, splice sites or the like. Furthermore, primers to repeated sequences can be used in isothermal amplification methods such as those set forth herein.

Further description of exemplary amplification methods that can be used in the invention can be found in U.S. Pat. No. 6,355,431 and U.S. Ser. No. 10/871,513.

A nucleic acid primer used in a method of the invention can have any of a variety of compositions or sizes, so long as it has the ability to hybridize to a template nucleic acid with sequence specificity and can participate in strand synthesis from the template. For example, a primer can be a nucleic acid having a native structure or an analog structure. A nucleic acid with a native structure generally has a backbone containing phosphodiester bonds and can be, for example, deoxyribonucleic acid or ribonucleic acid. An analog structure can have an alternate backbone including, without limitation, phosphoramide (see, for example, Beaucage et al., Tetrahedron 49(10):1925 (1993) and references therein; Letsinger, J. Org. Chem. 35:3800 (1970); Sprinzl et al., Eur. J. Biochem. 81:579 (1977); Letsinger et al., Nucl. Acids Res. 14:3487 (1986); Sawai et al, Chem. Lett. 805 (1984), Letsinger et al., J. Am. Chem. Soc. 110:4470 (1988); and Pauwels et al., Chemica Scripta 26:141 91986)), phosphorothioate (see, for example, Mag et al., Nucleic Acids Res. 19:1437 (1991); and U.S. Pat. No. 5,644,048), phosphorodithioate (see, for example, Briu et al., J. Am. Chem. Soc. 11 1:2321 (1989), O-methylphophoroamidite linkages (see, for example, Eckstein, Oligonucleotides and Analogues: A Practical Approach, Oxford University Press), and peptide nucleic acid backbones and linkages (see, for example, Egholm, J. Am. Chem. Soc. 114:1895 (1992); Meier et al., Chem. Int. Ed. Engl. 31:1008 (1992); Nielsen, Nature, 365:566 (1993); Carlsson et al., Nature 380:207 (1996)). Other analog structures include those with positive backbones (see, for example, Denpcy et al., Proc. Natl. Acad. Sci. USA 92:6097 (1995); non-ionic backbones (see, for example, U.S. Pat. Nos. 5,386,023, 5,637,684, 5,602,240, 5,216,141 and 4,469,863; Kiedrowshi et al., Angew. Chem. Intl. Ed. English 30:423 (1991); Letsinger et al., J. Am. Chem. Soc. 110:4470 (1988); Letsinger et al., Nucleoside & Nucleotide 13:1597 (1994); Chapters 2 and 3, ASC Symposium Series 580, “Carbohydrate Modifications in Antisense Research”, Ed. Y. S. Sanghui and P. Dan Cook; Mesmaeker et al., Bioorganic & Medicinal Chem. Left. 4:395 (1994); Jeffs et al., J. Biomolecular NMR 34:17 (1994); Tetrahedron Lett. 37:743 (1996)) and non-ribose backbones, including, for example, those described in U.S. Pat. Nos. 5,235,033 and 5,034,506, and Chapters 6 and 7, ASC Symposium Series 580, “Carbohydrate Modifications in Antisense Research”, Ed. Y. S. Sanghui and P. Dan Cook. Analog structures containing one or more carbocyclic sugars are also useful in the methods and are described, for example, in Jenkins et al., Chem. Soc. Rev. (1995) pp 169-176. Several other analog structures that are useful in the invention are described in Rawls, C & E News Jun. 2, 1997 page 35.

A further example of a nucleic acid primer with an analog structure that is useful in the invention is a peptide nucleic acid (PNA). The backbone of a PNA is substantially non-ionic under neutral conditions, in contrast to the highly charged phosphodiester backbone of naturally occurring nucleic acids. This provides two non-limiting advantages. First, the PNA backbone exhibits improved hybridization kinetics. Secondly, PNAs have larger changes in the melting temperature (Tₘ) for mismatched versus perfectly matched base pairs. DNA and RNA typically exhibit a 2-4° C. drop in Tₘ for an internal mismatch. With the non-ionic PNA backbone, the drop is closer to 7-9° C. This can provide for better sequence discrimination. Similarly, due to their non-ionic nature, hybridization of the bases attached to these backbones is relatively insensitive to salt concentration.

A nucleic acid primer useful in the invention can contain a non-natural sugar moiety in the backbone. Exemplary sugar modifications include but are not limited to 2′ modifications such as addition of halogen, alkyl, substituted alkyl, allcaryl, arallcyl, O-allcaryl or O-aralkyl, SH, SCH₃, OCN, Cl, Br, CN, CF₃, OCF₃, SOCH₃, SO₂, CH₃, NO2, N₃, NH₂, heterocycloallcyl, heterocycloallcaryl, aminoallcylamino, polyallcylamino, substituted silyl, and the like. Similar modifications can also be made at other positions on the sugar, particularly the 3′ position of the sugar on the 3′ terminal nucleotide or in 2′-5′ linked oligonucleotides and the 5′ position of a 5′ terminal nucleotide.

A nucleic acid primer used in the invention can include native or non-native bases. In this regard a native deoxyribonucleic acid can have one or more bases selected from the group consisting of adenine, thymine, cytosine or guanine and a ribonucleic acid can have one or more bases selected from the group consisting of uracil, adenine, cytosine or guanine. Exemplary non-native bases that can be included in a nucleic acid, whether having a native backbone or analog structure, include, without limitation, inosine, xathanine, hypoxathanine, isocytosine, isoguanine, 5-methylcytosine, 5-hydroxymethyl cytosine, 2-aminoadenine, 6-methyl adenine, 6-methyl guanine, 2-propyl guanine, 2-propyl adenine, 2-thioLiracil, 2-thiothymine, 2-thiocytosine, 15-halouracil, 15-halocytosine, 5-propynyl uracil, 5-propynyl cytosine, 6-azo uracil, 6-azo cytosine, 6-azo thymine, 5-uracil, 4-thiouracil, 8-halo adenine or guanine, 8-amino adenine or guanine, 8-thiol adenine or guanine, 8-thioalkyl adenine or guanine, 8-hydroxyl adenine or guanine, 5-halo substituted uracil or cytosine, 7-methylguanine, 7-methyladenine, 8-azaguanine, 8-azaadenine, 7-deazaguanine, 7-deazaadenine, 3-deazaguanine, 3-deazaadenine or the like. A particular embodiment can utilize isocytosine and isoguanine in a nucleic acid in order to reduce non-specific hybridization, as generally described in U.S. Pat. No. 5,681,702.

A non-native base used in a nucleic acid primer of the invention can have universal base pairing activity, wherein it is capable of base pairing with any other naturally occurring base. Exemplary bases having universal base pairing activity include 3-nitropyrrole and 5-nitroindole. Other bases that can be used include those that have base pairing activity with a subset of the naturally occurring bases such as inosine which base pairs with cytosine, adenine or uracil.

A nucleic acid primer having a modified or analog structure can be used in the invention, for example, to facilitate the addition of labels, or to increase the stability or half-life of the molecule under amplification conditions or other conditions, such as those set forth herein. As will be appreciated by those skilled in the art, one or more of the above-described nucleic acids can be used in the present invention, including, for example, as a mixture including molecules with native or analog structures. In addition, a nucleic acid primer used in the invention can have a structure desired for a particular amplification technique used in the invention.

In particular embodiments a nucleic acid primer useful in the invention can include a detection moiety. A detection moiety can be used, for example, to detect one or more concatenated nucleic acid targets using methods such as those set forth below. A detection moiety can be a primary label that is directly detectable or secondary label that can be indirectly detected, for example, via direct or indirect interaction with a primary label. Exemplary primary labels include, without limitation, an isotopic label such as a naturally non-abundant radioactive or heavy isotope; chromophore; luminophore; fluorophore; calorimetric agent; magnetic substance; electron-rich material such as a metal; electrochemiluminescent label such as Ru(bpy)₃²⁺; or moiety that can be detected based on a nuclear magnetic, paramagnetic, electrical, charge to mass, or thermal characteristic. Fluorophores that are useful in the invention include, for example, fluorescent lanthanide complexes, including those of Europium and Terbium, fluorescein, rhodamine, tetramethylrhodamine, eosin, erythrosin, coumarin, methyl-coumarins, pyrene, Malacite green, Cy3, Cy5, stilbene, Lucifer Yellow, Cascade Blue™, Texas Red, alexa dyes, phycoerythin, bodipy, and others known in the art such as those described in Haugland, Molecular Probes Handbook, (Eugene, Oreg.) 6th Edition; The Synthegen catalog (Houston, Tex.), Lakowicz, Principles of Fluorescence Spectroscopy, 2nd Ed., Plenum Press New York (1999), or WO 98/59066. Labels can also include enzymes such as horseradish peroxidase or alkaline phosphatase or particles such as magnetic particles or optically encoded nanoparticles.

Exemplary secondary labels include binding moieties. A binding moiety can be attached to a nucleic acid primer to allow detection or isolation via specific affinity for a receptor. Exemplary pairs of binding moieties and receptors that can be used in the invention include, without limitation, antigen and immunoglobulin or active fragments thereof, such as FAbs; immunoglobulin and immunoglobulin (or active fragments, respectively); avidin and biotin, or analogs thereof having specificity for avidin such as imino-biotin; streptavidin and biotin, or analogs thereof having specificity for streptavidin such as imino-biotin; carbohydrates and lectins; and other known proteins and their ligands. It will be understood that either partner in the above-described pairs can be attached to a nucleic acid primer, and the primer can be detected or isolated based on binding to the respective partner.

In a particular embodiment, the secondary label can be a chemically modifiable moiety. In this embodiment, labels having reactive functional groups can be incorporated into a nucleic acid. The functional group can be subsequently covalently reacted with a primary label. Suitable functional groups include, but are not limited to, amino groups, carboxy groups, maleimide groups, oxo groups and thiol groups.

A method of the invention can further include a step of signal amplification in which the number of detectable labels attached to a nucleic acid is increased. In one embodiment, a signal amplification step can include providing a nucleic acid that is labeled with a ligand having affinity for a particular receptor. A first receptor having one or more sites capable of binding the ligand can be contacted with the labeled nucleic acid under conditions where a complex forms between the receptor and ligand-labeled nucleic acid. Furthermore, the receptor can be contacted with a signal amplification reagent that has affinity for the receptor. The amplification reagent can be, for example, the ligand, a mimetic of the ligand, or a second receptor having affinity for the first receptor. The amplification reagent can in turn be labeled with the ligand such that a multimeric complex can form between the ligand receptor and signal amplification reagent. The presence of the multimeric complex can then be detected, for example, by detecting the presence of a detectable label on the receptor or the signal amplification reagent. The components included in a signal amplification step can be added in any order so long as a detectable complex is formed. Furthermore, other binding moieties and binding partner pairs such as those set forth herein previously can be used for signal amplification.

In an exemplary embodiment, signal amplification can be carried out using a nucleic acid labeled by streptavidin-phycoerythrin (SAPE) and a biotinylated anti-SAPE antibody. For example, a three step protocol can be employed in which probes that have been modified to incorporate biotin are first incubated with streptavidin-phycoerythrin (SAPE), followed by incubation with a biotinylated anti-streptavidin antibody, and finally incubation with SAPE again. However, the reagents can be combined in any of a variety of desired orders. This process creates a cascading amplification sandwich since streptavidin has multiple antibody binding sites and the antibody has multiple biotins. Those skilled in the art will recognize from the teaching herein that other receptors such as avidin, modified versions of avidin, or antibodies can be used in an amplification complex and that different labels can be used such as Cy3, Cy5 or others set forth previously herein. Further exemplary signal amplification techniques and components that can be used in the invention are described, for example, in U.S. Pat. No. 6,203,989 B1.

As a further example, signal amplification can be carried out using a nucleic acid labeled by anti-dinitrophenyl rabbit IgG labeled with Alexa 647 (anti-DNP rIgG-Alexa 647) and a goat anti-rabbit IgG labeled with dinitrophenyl (anti-rabbit gIgG-DNP). A three step protocol similar to that described above for SAPE can be employed. This process, or one in which reagents are combined in different ways can be used to create a cascading amplification sandwich. Those skilled in the art will recognize that the antibody species and labels exemplified above can be replaced with appropriate antibodies from other sources or with different labels, respectively. Two or more signal amplification systems can be used to detect two or more labels on a particular probe location such as a probe feature or bead of an array. For example, the order of sequence portions in a first and second concatenated nucleic acid target can be identified by simultaneous detection of SAPE and DNP signals on particular probes, for example, in an array.

Binding moieties can be particularly useful when attached to primers used for amplification of a template nucleic acid because the amplified product can be attached to an array via the binding moieties. Furthermore, binding moieties can be useful for separating amplified products from other components of an amplification reaction, concentrating the amplified products, or detecting the amplified products.

A label such as those set forth above can be added to a probe or primer for detection of a particular target.

A nucleic acid primer used in a method of the invention can include a complementary sequence that is any length capable of binding to a desired template with sufficient stability and specificity to prime polymerase replication activity. The complementary sequence can include all or a portion of a primer used for amplification. For example, a primer can include a 3′ region that is complementary to a target genomic DNA sequence and a 5′ region that is not complementary to the gDNA, rather having an address sequence or universal priming site in accordance with embodiments set forth in further detail below. Amplification can be carried out with primers having relatively short complementary sequences including, for example, at most 5, 6, 7, 8, 9, 10, 11, 12, 13, 14, 15, 16, 17, 18, 19, 20, 25, 30, 35, 40, 50, 60, 70, 80, 90, 100, 200, 300, 400, 500 nucleotides in length. Those skilled in the art will recognize that specificity of hybridization is generally increased as the length of the nucleic acid primer is increased. Thus, a longer nucleic acid primer can be used, for example, to increase specificity or reproducibility of replication, if desired. Accordingly, a primer used in a method of the invention can be at least 5, 6, 7, 8, 9, 10, 11, 12, 13, 14, 15, 16, 17, 18, 19, 20, 25, 30, 35, 40, 50, 60, 70, 80, 90, 100, 200, 300, 400, 500 or more nucleotides long.

Typically, amplification is carried out with a population of nucleic acid primers that hybridizes to different portions of a nucleic acid template. A population of primers used in the invention can include members having a random or semi-random complement of sequences. Thus, a population of primers can have members with a fixed sequence length in which one or more positions along the sequence are randomized within the population. By way of example, a population of 12mer primers can have a sequence that is identical except at one particular position, say position 5, where any of the four native DNA nucleotides are incorporated, thereby producing a population having four different primer members. In a particular embodiment, multiple positions along the sequence can be combinatorially randomized. For example, a nucleic acid primer can have 2, 5, 10, 15, 20, 25, 30, 35, 40, 50, 60, 70, 80, 90, 100 or more positions that are randomized. For example a 12mer primer that is randomized at each position with 4 possible native DNA nucleotides can contain up to 4¹²=1.7×10⁷ members.

In particular embodiments, a population of primers used in the invention can include members with sequences that are designed based on rational algorithms or processes. Similarly, a population of nucleic acids can include members each having at least a portion of their sequence designed based on rational algorithms or processes. Rational design algorithms or processes can be used to direct synthesis of a nucleic acid product having a discrete sequence or to direct synthesis of a nucleic acid mixture that is biased to preferentially contain particular sequences.

Using rational design methods, sequences for nucleic acids in a population can be selected, for example, based on known sequences in the nucleic acid template that are to be amplified or detected. The sequences can be selected such that the population preferentially includes sequences that hybridize to the template with a desired coverage. For example, a population of primers can be designed to preferentially include members that hybridize to a particular chromosome or particular portion of a genome such as coding regions or non coding regions.

Primers useful in the invention can be designed to preferentially omit or reduce sequences that hybridize to particular sequences in a genome such as known repeats or repetitive elements including, for example, Alu repeats. Accordingly, a single primer, such as one used in arbitrary-primer amplification, can be designed to include or exclude a particular sequence. Similarly a population of primers, such as a population used for random primer amplification, can be synthesized to preferentially exclude or include particular sequences such as Alu repeats. A population of random primers can also be synthesized to preferentially include a higher content of G and/or C nucleotides compared to A and/or T nucleotides. A random primer population that is GC rich will have a higher probability of hybridizing to high GC regions of a genome such as gene coding regions of a human genome which typically have a higher GC content than non-coding regions. Conversely, AT rich primers can be synthesized to preferentially amplify or anneal to AT rich regions such as non-coding regions of a human genome. Other parameters that can be used to influence primer design include, for example, preferential removal of sequences that render primers self complementary, prone to formation of primer dimers or prone to hairpin formation or preferential selection of sequences that have a desired maximum, minimum or average Tₘ. Exemplary methods and algorithms that can be used in the invention for designing and synthesizing probes include those described in US 2003/0096986A1.

Primers in a population of random primers can have a region of identical sequence such as a universal tail. A universal tail can include a universal priming site for a subsequent amplification step. Thus, a population of primers can have a region where their sequences differ from each other and a second region that is the same for several of the primers, the latter region forming a universal priming site for a universal primer. Each primer can also include a region that anneals to a particular binding agent useful for isolating or detecting amplified sequences such as a probe in a universal array. Methods for making and using a population of random primers with universal tails are described, for example, in Singer et al., Nucl. Acid. Res. 25:781-786 (1997) or Grothues et al., Nucl. Acids Res. 21:1321-2 (1993).

A variety of hybridization conditions can be used in the present invention, such as high, moderate or low stringency conditions including, but not limited to those described in Sambrook et al., supra, (2001) or in Ausubel et al., supra, (1998). Stringent conditions favor specific sequence-dependent hybridization. In general, longer sequences and increased temperatures favor specific sequence-dependent hybridization. A useful guide to the hybridization of nucleic acids is found in Tijssen, Techniques in Biochemistry and Molecular Biology—Hybridization with Nucleic Acid Probes, “Overview of principles of hybridization and the strategy of nucleic acid assays” (1993).

Amplification and detection steps used in the invention are generally carried out under stringency conditions which selectively allow formation of a hybridization complex in the presence of complementary sequences. Stringency can be controlled by altering a step parameter that is a thermodynamic variable, including, but not limited to, temperature, formamide concentration, salt concentration, chaotropic salt concentration, pH, organic solvent concentration, or the like. These parameters can also be used to control non-specific binding, as is generally outlined in U.S. Pat. No. 5,681,697. Thus, if desired, certain steps can be performed under relatively high stringency conditions to reduce non-specific binding.

Those skilled in the art will recognize that any of a variety of nucleic acids used in the invention, such as probes, can have one or more of the properties set forth above and exemplified with respect to primers. Furthermore, probes can be made or used using methods similar to those employed for primers.

The type of polymerase and conditions used for amplification in a method of the invention can be chosen to obtain one or more products having a desired length. In particular embodiments, relatively short amplification products can be obtained in a method of the invention, for example, by amplifying a concatenated nucleic acid, or other template nucleic acid, with a polymerase of low processivity or by fragmenting the template or its amplification products with a nucleic acid cleaving agent such as an endonuclease or chemical agent. Accordingly, a method of the invention can be used to obtain amplified products that are, without limitation, at most about 10 kb, 5 kb, 4 kb, 3 kb, 2 kb, 1 kb, 0.8 kb, 0.6 kb, 0.5 kb, 0.4 kb, 0.2 kb, or 0.1 kb in length.

In alternative embodiments, a method of the invention can be used to produce relatively large amplified products. In accordance with such embodiments, a method of the invention can be used to obtain amplified products that are at least about 10 kb, 15 kb, 20 kb, 25 kb, 30 kb in length.

A low processivity polymerase can synthesize less than 100 bases per polymerization event. Shorter fragments can be obtained if desired by using a polymerase that synthesizes less than 50, 40, 30, 20, 10 or 5 bases per polymerization event under the conditions of amplification. Exemplary polymerases that are capable of low processivity and useful for amplifying gDNA in the invention include, without limitation, Taq polymerase, T4 polymerase, “monomeric” E. coli Pol III (lacking the beta subunit), or E. coli DNA Pol I or its 5′ nuclease deficient fragment known as Klenow polymerase. A non-limiting advantage of using a low processivity polymerase for amplification is that relatively small amplification products are obtained, thereby allowing efficient hybridization to nucleic acid arrays. If desired, small fragments can also be obtained by fragmenting an amplified nucleic acid product.

The invention further provides embodiments in which amplification occurs under conditions where the template is not globally denatured. An exemplary condition is a temperature at which the template remains substantially double stranded until contacted by other reagents. Such conditions are typically referred to as isothermal conditions. A concatenated nucleic acid, or other template nucleic acid, can be amplified under isothermal conditions using a polymerase having strand displacing activity. Exemplary polymerases that are capable of strand displacement include, without limitation, E. coli Pol I, exo⁻ Klenow polymerase, phi29, Bst DNA polymerase, or sequencing grade T7 exo− polymerase (such as SEQUENASE™ version 2.0 (USB, Cleveland, Ohio) which lacks the 28 amino acid region Lys118 to Arg 145 of T7 pol).

Further examples of polymerases that are useful in the invention include, without limitation, bacteriophage phi29 DNA polymerase (U.S. Pat. Nos. 5,198,543 and 5,001,050), exo(−)Bca DNA polymerase (Walker and Linn, Clinical Chemistry 42:1604-1608 (1996)), phage M2 DNA polymerase (Matsumoto et al., Gene 84:247 (I 989)), phage phiPRD 1 DNA polymerase (Jung et al., Proc. Natl. Acad. Sci. USA 84:8287 (1987)), exo(−)VENT™ DNA polymerase (Kong et al., J Biol. Chem. 268.1965-1975 (1993)), T5 DNA polymerase (Chatterjee et al., Gene 97:13-19 (1991)), and PRD1 DNA polymerase (Zhu et al., Biochim. Biophys. Acta. 1219:267-276 (1994)). The invention can also be carried out with variants of the above-described polymerases, so long as they retain polymerase activity. Exemplary variants include, without limitation, those that have decreased exonuclease activity, increased fidelity, increased stability or increased affinity for one or more nucleoside analogs.

A further polymerase variant that is useful in a method of the invention is a modified polymerase that, when compared to its wild type unmodified version, has a reduced or eliminated ability to add non-template directed nucleotides to the 3′ end of a nucleic acid. Exemplary variants include those that affect activity of the polymerase toward adding all types of nucleotides or one or more specific types of nucleotides such as pyrimidine nucleotides, purine nucleotides, A, C, T, U or G. Examples of modified polymerases having reduced or eliminated ability to add non-template directed nucleotides to the 3′ end of a nucleic acid are described, for example, in U.S. Pat. No. 6,306,588 or Yang et al., Nucl. Acids Res. 30:4314-4320 (2002). In a particular embodiment, such a polymerase variant can be used in an SBE or ASPE detection method described in further detail below.

Generally, polymerase activity, including, for example, processivity and strand displacement activity, can be influenced by factors such as pH, temperature, ionic strength, and buffer composition. Those skilled in the art will know which types of polymerases and conditions can be used to obtain amplification products having a desired length in view of that which is known regarding the activity of the polymerases as described, for example, in Eun, Enzymology Primer for Recombinant DNA Technology, Academic Press, San Diego (1996) or will be able to determine appropriate polymerases and conditions by systematic testing using known assays, such as gel electrophoresis or mass spectrometry, to measure the length of amplified products.

In particular embodiments of the invention, a DNA target can be in vitro transcribed into an RNA target. This can offer several non-limiting advantages when used, for example, in combination with a primer extension assay including, for example, a solid phase primer extension assay performed in an array. Primer extension typically includes a step of hybridizing a DNA probe to a DNA target and subsequently modifying the probe-target hybrid with a DNA polymerase. Under some conditions these assays can be compromised by artifacts arising from unwanted formation of probe-probe hybrids, for example, due to physical proximity on an array surface, which can lead to ectopic extension of the probe-probe hybrids. In embodiments of the invention where RNA targets are hybridized to DNA probes, such artifacts can be avoided because DNA polymerase is replaced with reverse transcriptase (RT) which is selective for hybrids having an RNA template and does not efficiently modify or extend DNA-DNA probe hybrids.

Exemplary methods for in vitro transcription of a DNA template and detection of the RNA product are described in U.S. Ser. No. 10/871,513. For example, a template DNA can be random-primer amplified using a population of primers including a random 3′ domain and a 5′ domain having an RNA polymerase promoter sequence. The amplified products having an RNA polymerase promoter can be in vitro transcribed to RNA form using a T7 RNA polymerase and a complementary T7 primer. The RNA product can then be detected using DNA probes.

If desired, the complexity can be reduced for a population of nucleic acids made or used in accordance with the invention. Complexity reduction can be carried out by removing particular sequences from a population of gDNA fragments or a population of products amplified from a concatenated nucleic acid template. In particular embodiments, nucleic acid targets having high copy number can be inhibited from hybridizing to capture probes by removal or sequestration of the high copy number nucleic acids. For example, Cot analysis can be used in which abundant species are kinetically driven to reanneal while leaving the single copy species in a single stranded state capable of hybridization to probes. Thus, a population of nucleic acids can be treated with cot oligonucleotides that are complementary to particular repeated sequences, or to other sequences that are desired to be titrated out of the sample, prior to exposure of the sample to an array of probes. In another example, a sample having high copy number sequences can be cooled to a temperature and for a time period that is sufficiently short for a substantial fraction of over-represented sequences to re-anneal but insufficient for substantial re-annealing of sequences present in low copy numbers. The resulting sample will have a reduced amount of repeated sequences available for subsequent interaction with an array of probes.

Undesired fragments that form double stranded species, for example, in Cot analysis or reannealing, can be separated from single stranded species based on different properties of single and double stranded nucleic acids. In a particular embodiment, enzymes that preferentially cleave double stranded DNA can be used. For example, DNAse I can cleave double-stranded DNA 100 to 500 fold faster than single stranded DNA under known conditions. Accordingly, undesired fragments can be removed by treatment with Cot oligonucleotides or by fragment reannealing, and treatment with DNAse I under conditions wherein undesired fragments preferentially form double stranded species and get cleaved. Furthermore, other enzymes that preferentially modify, cleave or bind to double stranded species compared to single stranded species can be used to separate the species in a method of the invention such as sequence specific restriction endonucleases or Kamchatka crab duplex-specific endonuclease. Complexity reduction can also be carried out by selective amplification of a subset of sequence portions in a nucleic acid sample using methods known in the art such as those described in U.S. Ser. No. 10/871,513.

Following replication of a concatenated nucleic acid, or other nucleic acid, amplification products can be separated from unmodified nucleic acids such as unreacted primers or the template. For example, it can be desirable to remove unextended or unreacted primers if unextended primers will compete with the extended or labeled primers in the detection methods that are to be used. Any of a number of different techniques can be used to facilitate the removal of unextended primers. While the discussion below is directed to amplification reactions for clarity, it will be understood that these techniques can also be used to separate modified and unmodified nucleic acids in a detection step.

Separation of nucleic acids can be mediated by selective incorporation of a label including, for example, one or more of the primary or secondary labels described previously herein. A nucleic acid can be conveniently labeled in a method of the invention by a moiety introduced during an amplification or modification reaction via a labeled primer, labeled nucleotide precursor or both. In particular embodiments, one or more NTPs (deoxynucleotidetriphosphates or nucleotidetriphosphates) used to replicate a nucleic acid can include a secondary detectable label that can be used to separate modified primers from unmodified primers lacking the label. Nucleic acids having an incorporated secondary label can be separated from those lacking the label based, for example, on binding to a receptor having specificity for the label. The receptor can be attached, for example, to a solid phase substrate. Primary labels can be used to separate nucleic acids in a chromatographic or sorting method. Similarly, nucleic acids having an incorporated secondary label can be separated from those lacking the label in a chromatographic or sorting method based on detection of a receptor that provides a primary label to the nucleic acid-receptor complex. Sorting can be carried out, for example, on particles in a flow cytometer or fluorescent activated cells sorter. Chromatographic separation can be accomplished using standard size exclusion resins such as G-50 resin, reverse phase media or ion exchange media. Other useful separation methods include, without limitation, ultrafiltration such as with Amicon or Centricon columns, or ethanol-like precipitation methods.

In embodiments, including attachment of a binding partner to a solid support, the solid support can be selected, for example, from those described herein with respect to detection arrays. Particularly useful substrates include, for example, magnetic beads which can be easily introduced to the nucleic acid sample and easily removed with a magnet. Other known affinity chromatography substrates can be used as well. Known methods can be used to attach a binding partner to a solid support.

A concatenated nucleic acid or amplified product produced from a concatenated nucleic acid template can be treated to produce fragments if desired. A single-stranded or double stranded nucleic acid can be fragmented by any of a variety of physical, chemical or biochemical methods known in the art. For example, a double-stranded nucleic acid can be digested with an endonuclease using methods such as those set forth above in regard to cleaving gDNA. Typically, a concatenated nucleic acid target will be cleaved with an RE that recognizes a different recognition site than the site that wa cleaved in a gDNA to produce fragments for concatenation.

Non-specific endonucleases can also be used to produce genome fragments of a desired average size. Because the endonuclease reaction is bi-molecular, the rate of fragmentation can be manipulated by altering conditions such as the concentrations of the endonuclease, DNA or both. Specifically, a reduction in the concentration of either endonuclease, DNA or both can be used to reduce reaction rate resulting in increased average fragment sizes. Increasing concentrations of either endonuclease, DNA recognition sequence or both will allow for increased efficiency, approaching maximum velocity (Vₘₐₓ) for the particular enzyme leading to reduced average fragment sizes. Similar changes in conditions can also be applied to site-specific endonucleases because their reactions with DNA are also bi-molecular. Other reaction conditions can also affect the rate of cleavage including, for example, temperature, salt concentration and time of reaction. Methods for altering nuclease reaction rates to produce polynucleotide fragments of determined average size are described, for example, in Sambrook et al., supra, (2001) or in Ausubel et al., supra, (1998).

In a further embodiment, nucleic acid fragments can be produced by cleaving a concatenated nucleic acid or amplified product that contains an exogenous base. In a particular embodiment, amplified products made in a method of the invention can have bases exogenous to native DNA at various positions such as the positions for one or more of A, T, C or G. For example, bases exogenous to native DNA can replace all or a portion of the A, T, C or G bases in an amplified product. Typically, not all of the A, T, C or G bases are replaced with an exogenous base. For example, a nucleic acid made in a method of the invention can include, on average, exogenous bases present in at least about 0.1%, 1%, 2%, 3%, 4% or 5%, at most about 10%, 15%, 20% or 25% of the positions for A, T, G or C or in a range between these values.

Nucleic acids having bases exogenous to native DNA can be produced, for example, by incorporation of a nucleotide having the exogenous base. Such a nucleotide can be incorporated by an enzyme such as a polymerase or ligase. In particular embodiments, an exogenous base can be introduced into the products of an amplification reaction, for example, by spiking into the reaction one or more type of nucleotide having the exogenous base. Useful ranges of nucleotide having an exogenous base that can be spiked into a reaction include, but are not limited to, at least about 0.1%, 1%, 2%, 3%, 4% or 5%, at most about 10%, 15%, 20% or 25%, or a range between these values wherein the percentages refer to the amount of exogenous base-containing nucleotide spiked into the reaction compared to the total amount of polymerase-reactive nucleotide in the reaction. Higher percentages can be used, such as at least about 10%, 15%, or 20% if desired, for example, to achieve higher levels of incorporation or to accommodate an exogenous base that has a relatively low level of incorporation during amplification in the presence of native bases. Conversely, lower percentages of nucleotide having an exogenous base can be used, such as at most about 1%, 2%, 3%, 4% or 5% if desired, for example, to achieve lower levels of incorporation or to accommodate an exogenous base that has a relatively high level of incorporation during amplification in the presence of native bases. Although the above ranges are exemplified in terms of spiking in a single type of exogenous base-containing nucleotide into a reaction, it will be understood that the ranges can also be applied to the total amount of exogenous base-containing nucleotides present in a reaction when two or more different types are spiked into the reaction.

Nucleic acids having an exogenous base can be fragmented by a reagent that cleaves the fragments in a manner dependent upon the presence of the exogenous base. Typically, such a reagent will cleave the phosphodiester backbone of the nucleic acid upon binding to the exogenous base or will modify the exogenous base such that the phosphodiester backbone can be cleaved. For example, a DNA having exogenous uracil present can be cleaved by uracil DNA glycosylase (UDG) which removes the uracil base, followed by heating or chemical methods which cleave the abasic site. Uracil can be incorporated into genomic fragments and the uracil-containing genomic fragments cleaved as described, for example, in U.S. Pat. No. 5,952,176; 6,090,553 or 6,440,705.

In a further embodiment, bases in a nucleic acid can be modified to a form that is recognized and cleaved by 8-hydroxyguanine DNA glycosylase (FPG protein). Exemplary modifications that can be recognized by FPG protein include, without limitation, 8-hydroxyguanine; imidazole ring-opened derivatives of adenine or guanine, designated 4,6-diamino-5-formamidopyrimidine and 2,6-diamino-4-hydroxy-5-formamidopyrimidine, respectively; N7-methylformamidopyrimidines; 5-hydroxyuracil; or 5-hydroxycytosine. Nucleic acids can be modified to include an FPG recognized base and cleaved with FPG using methods known in the art as described, for example, in U.S. Pat. No. 6,048,696.

A method of the invention can use chemical cleavage methods to produce fragments of a nucleic acid, such as a concatenated nucleic acid or an amplified product of a concatenated nucleic acid. Cleavage can be carried out with a reagent that reacts with a native nucleic acid. Alternatively, a nucleic acid can be produced or modified to contain exogenous bases that are more susceptible to chemical cleavage compared to native bases such that a product containing the exogenous base is preferentially cleaved over a nucleic acid lacking the exogenous base. Exemplary exogenous bases and reagents for cleaving the bases include, but are not limited to, 7-deaza-7-nitro-dATP, 7-deaza-7-nitro-dGTP, 5-hydroxy-dCTP and 5-hydroxy-dUTP which can be preferentially cleaved by treatment with an oxidant followed by an organic base as described, for example, in Wolfe et al. Proc. Natl. Acad. Sci. USA 99:11073-11078 (2002). Further exogenous bases and cleavage methods that can be used in a method of the invention include, for example, those employed in Maxam-Gilbert DNA sequencing (see, for example, Maxam et al., Methods Enz. 65:499-560 (1980)) or in Eckstein sequencing (see, for example, Nakamaye et al., Nucleic Acids Res. 16:9947-9959 (1988)). Other methods that can be used to produce fragments include, for example, treatment with chemical agents that disrupt the phosphodiester backbone of DNA such as those that cleave bonds by a free radical mechanism, UV light, mechanical disruption or the like.

If desired, fragments of a desired average size can be produced by titrating the amount of cleavage reagent used or controlling the duration of the cleavage reaction or both. In a particular embodiment, a population of genome fragments having exogenous bases can be reacted with a cleavage reagent under conditions wherein the reaction does not go to completion.

Two or more sequence portions from a gDNA that are re-ordered about at least one restriction site in a concatenated nucleic acid sequence can be identified to determine the location of the restriction site(s) in the gDNA. Re-ordered sequence portions can be identified by determining the sequence for the concatenated nucleic acid, or a product amplified using the concatenated nucleic acid as template, and comparing it to the genomic sequence. A sequence of the concatenated nucleic acid, or its amplified product, can be determined using a nucleic acid sequencing method known in the art. Exemplary sequencing methods include, without limitation, the Sanger dideoxy sequencing method, Maxam-Gilbert sequencing, pyrosequencing and modified versions of these methods.

Sequence portions of a gDNA that are re-ordered about a restriction site in a concatenated nucleic acid can also be determined using a probe that has different binding affinity for one or more re-ordered portions in a concatenated nucleic acid target compared to its affinity for a reference nucleic acid, such as the gDNA. A concatenated nucleic acid target is understood to include the product of a genome fragment ligation reaction, or a product of amplification using such a ligation product as a template. A particularly useful probe for detecting a concatenated nucleic acid target is a nucleic acid probe. For example, a nucleic acid probe that is complementary to a target sequence that spans adjacent portions across an RE recognition sequence in a gDNA will have higher affinity for the gDNA compared to a concatenated DNA in which the portions are no longer adjacent. Thus, re-ordering can be identified according to a decreased amount of concatenated target bound to the probe or decreased kinetic rates for binding between the probe and target. Those skilled in the art will understand that in addition to nucleic acid probes, other types of probes capable of differentiating sequence differences in two or more nucleic acids can also be used such as sequence specific nucleic acid binding proteins.

In a further embodiment, sequence portions of a gDNA that are re-ordered about a restriction site in a concatenated nucleic acid can be determined based on differential modification of a probe in the presence of the concatenated nucleic acid target compared to in the presence of a reference nucleic acid, such as the gDNA. For example, as shown in FIG. 1, a nucleic acid probe that is complementary to a target sequence that spans adjacent portions across an RE recognition sequence in a gDNA and that has a free 3′ OH can be extended in a template dependent fashion by a polymerase in the presence of a concatenated nucleic acid target having the adjacent portions. Thus, signal generated from the modified probe indicates that the RE recognition site is not reactive (i.e. the site is methylated in the example of FIG. 1). However, continuing with the Example of FIG. 1, probe modification is inhibited when the probe is hybridized to a concatenated DNA target in which the portions are no longer adjacent. Such inhibition or absence of probe modification indicates that restriction occurred for the gDNA at the restriction site (i.e. that the RE recognition site is not methylated).

A probe modification reaction used in a method of the invention can include, without limitation, allele specific primer extension (ASPE), single base extension (SBE), ligation, extension and ligation, or pyrosequencing. Other useful methods include Invader™-based methods, cycling probe technology and sandwich assays. Such methods can be carried out as set forth below or as further described, for example, in U.S. Pat. No. 6,355,431 B1, US Pat. App. Pub. No. US 2003/0211489 or U.S. Ser. No. 10/871,513.

Extension assays are generally carried out by modifying the 3′ end of a first nucleic acid when hybridized to a second nucleic acid. The second nucleic acid can act as a template directing the type of modification made to the first. In the case of polymerase-based extension, base pairing interactions can direct the incorporation of one or more nucleotides. Polymerase extension assays are particularly useful, for example, due to the relative high-fidelity of polymerases and their relative ease of implementation. Exemplary extension assays include, for example, ASPE, SBE or pyrosequencing and can be carried out using polymerases such as those set forth herein in regard to amplification methods.

Briefly, SBE utilizes an extension probe that hybridizes to a target nucleic acid, such as a concatenated nucleic acid target, at a location that is proximal or adjacent to a detection position, such as an RE recognition site. A polymerase can be used to extend the 3′ end of the probe with a nucleotide analog labeled with a detection label such as those described herein. Based on the fidelity of the enzyme, a nucleotide is only incorporated into the extension probe if it is complementary to the detection position in the target nucleic acid. If desired, the nucleotide can be derivatized such that it is chain terminating, and thus the probe is only modified with a single nucleotide. An exemplary chain terminating nucleotide analog useful for SBE detection is a dideoxynucleoside-triphosphate (also called dideoxynucleotides or ddNTPs, i.e. ddATP, ddTTP, ddCTP and ddGTP).

In a particular embodiment, a mixed SBE reaction can be run with two, three or four different nucleotides, each with a different label. Alternatively, discrete reactions can be run each with a different labeled nucleotide. For example, an SBE reaction can be carried out using a subset of nucleotides that lacks substantial amounts of one or more of A, C, T or G bases.

A nucleotide used in an SBE detection method can include any of a variety of known labels such as a primary or secondary detectable label described herein. The presence of the labeled nucleotide in the extended probe can be detected, for example, at a particular location in an array and the added nucleotide identified to determine the order of sequence portions about an RE recognition site. SBE can be carried out under known conditions such as those described in U.S. Ser. No. 10/871,513; Syvanen et al., Genomics 8:684-692 (1990); Syvanen et al., Human Mutation 3:172-179 (1994); U.S. Pat. Nos. 5,846,710 and 5,888,819; or Pastinen et al., Genomics Res. 7(6):606-614 (1997).

ASPE is an extension assay that utilizes extension probes that differ in nucleotide composition at their 3′ end. Briefly, ASPE can be carried out by hybridizing a target, such as a concatenated nucleic acid target, to an extension probe having a 3′ sequence that is complementary to a detection position, such as an RE recognition site or adjacent sequence portions of the concatenated nucleic acid. Template directed modification of the 3′ end of the probe, for example, by addition of a labeled nucleotide by a polymerase, yields a labeled extension product if the template includes the target sequence. The presence of such a labeled primer-extension product can then be detected to identify presence of a target having the detection position. It will be understood that ASPE need not be used to detect different alleles, but can be used to identify any of a variety of sequence differences whether or not they are identified as different alleles or associated with different alleles.

An exemplary embodiment of the invention utilizing ASPE detection is shown in FIG. 1. As shown, an ASPE probe can include a 5′ region that is complementary to a first sequence portion and a 3′ region that is complementary to a second sequence portion, wherein the first and second sequence portions are adjacent in a gDNA sequence. Sequence regions that are re-ordered in a concatenated nucleic acid target, although able to bind to the ASPE probe, do not support extension of the probe. However, sequence regions that are in the same order in the gDNA as in the concatenated nucleic acid target are able to hybridize to the probe such that the probe can be extended. Thus, incorporation of labeled nucleotides (indicated by asterisks in FIG. 1) indicates that sequence portions complementing the probe are not re-ordered and, accordingly, that the restriction site between the sequence portions was not reactive in the restriction step (i.e. having a methylated CpG in the Example of FIG. 1).

In particular embodiments, an ASPE reaction can include a nucleotide analog that is derivatized to be chain terminating. Thus, an ASPE probe in a probe-fragment hybrid can be modified to incorporate a single nucleotide analog without further extension. Exemplary chain terminating nucleotide analogs include, without limitation, those set forth above in regard to the SBE reaction. Furthermore, one or more nucleotides used in an ASPE reaction whether or not they are chain terminating can include a detection label such as those described previously herein.

Pyrosequencing is an extension assay that can be used to add one or more nucleotides to the 3′ end of a probe. Identification of a sequence in a probe-bound target is based on detection of a reaction product, pyrophosphate (PPi), produced during the addition of a dNTP to an extended probe, rather than on detection of a label attached to the nucleotide. One molecule of PPi is produced per dNTP added to the extension primer. Thus, by running sequential reactions with different nucleotides, and monitoring the reaction products, the identity of the added base can be determined. Accordingly, a probe that hybridizes to a first sequence portion can be extended into an adjacent portion and the sequence read to determine the order of the sequence portions. Pyrosequencing can be used in the invention using conditions such as those described in US 2002/0001801.

Another probe modification that can occur differentially to identify re-ordering in a concatenated nucleic acid target is ligation. For example, two nucleic acid probes that are complementary to adjacent sequence portions will be capable of being ligated if the sequence portions are adjacent in the concatenated nucleic acid target. However, if the portions ligated by the two probes are not adjacent in the concatenated nucleic acid target then the probes are not able to ligate. Thus, presence of a ligated product indicates that sequence portions of the gDNA are not re-ordered and, accordingly, that the restriction site between the sequence portions was not reactive in a restriction step used to generate gDNA fragments for concatenation. If desired, a ligation probe can be extended by a polymerase prior to ligation.

As will be appreciated by those in the art, the configuration of a probe modification reaction can take on any of several forms. In particular embodiments, the reaction can be done in solution, and then the modified probes can be detected. In one example of a solution based assay, probes modified in solution can be detected based on hybridization to capture probes, for example, on an array surface. If desired, a solution based assay can include amplification of a modified primer, followed by detection of the amplified product. In embodiments where amplification is used, probes that are modified in the presence of a concatenated nucleic acid target can include primer binding sites as exemplified below for oligonucleotide ligation amplification (OLA) and the GoldenGate™ assay. Alternatively, a probe modification reaction can occur on a surface. For example, a surface-immobilized capture probe can hybridize to a concatenated target nucleic acid such that the probe is modified on the surface. This and other probe modification methods can be carried out for arrayed probes as set forth in further detail below.

Detection with OLA involves the template-dependent ligation of two smaller probes into a single long probe, using adjacent portions of a concatenated nucleic acid target as the template. A first OLA probe and a second OLA probe can be hybridized to complementary sequences of the respective sequence portions. The two OLA probes are then covalently attached to each other to form a modified probe. In embodiments where the probes hybridize directly adjacent to each other, covalent linkage can occur via a ligase. Alternatively, an extension ligation (GoldenGate™) assay can be used wherein hybridized probes are non-contiguous and one or more nucleotides are added to at least one of the probes followed by ligation to join the probes via the added nucleotides. The probes can further include universal priming sites allowing the ligated products that were added by extension to be amplified by PCR. The PCR priming sites can flank the portions that hybridized to the target. The priming sites can further flank an address sequence that is specific for a capture probe on a: universal array. Exemplary conditions for OLA, GoldenGate™, amplification of modified probes and detection of address sequences using universal arrays, that are useful in the invention are described in U.S. Pat. No. 6,355,431 B1 and US Pat. App. Pub. No. 2003/0211489.

Adjacent sequence portions can be detected in a method of the invention using a circular probe, for example, in a rolling circle amplification (RCA) or padlock probe detection method. In a first embodiment, two ends of a single probe can be hybridized to a concatenated nucleic acid target. The probe can be ligated, or extended and ligated to form a circle. In the case of a padlock probe, the circle can be restricted to form a linear template for amplification as described, for example, in US Pat. App. Pub. No. 2004/0101835. In the case of RCA, addition of a polymerase and primer results in extension of the circular probe as described, for example, in Baner et al. (1998) Nuc. Acids Res. 26:5073-5078; Barany, F. (1991) Proc. Natl. Acad. Sci. USA 88:189-193; Lizardi et al. (1998) Nat Genet. 19:225-232; U.S. Pat. No. 6,355,431 B1 or US Pat. App. Pub. No. 2003/0211489.

If desired, a probe that is not part of a probe-fragment hybrid can be selectively modified compared to a probe-fragment hybrid. Selective modification of non-hybridized probes can be used to increase assay specificity and sensitivity, for example, by removing probes that are labeled in a template independent manner during the course of a polymerase extension assay. A particularly useful selective modification is degradation or cleavage of single stranded probes that are present in a population or array of probes following contact with target fragments under hybridization conditions. Exemplary enzymes that degrade single stranded nucleic acids include, without limitation, Exonuclease 1 or lambda Exonuclease.

As set forth above, the invention can be used to detect one or more reactive RE recognition sites. In particular, the invention is well suited to detection of a plurality of reactive RE recognition sites because the methods allow adjacent sequence portions of individual concatenated nucleic acid targets to be distinguished within large and complex pluralities. Individual sequence portions can be distinguished in the invention based on formation of probe-target hybrids and detection of physically separated probe-target hybrids. Physical separation of probe-target hybrids can be achieved by binding the hybrids or their components to one or more substrates. In particular embodiments, a probe-target hybrid can be distinguished from other probes and targets in a plurality based on the physical location of the hybrid on the surface of a substrate such as an array. A probe-target hybrid can also be bound to a particle. Particles can be discretely detected based on their location and distinguished from other probes and targets according to discrete detection of the particle in an array such as a solid-phase array in which particles are located at discrete locations on a surface or in a fluid array in which particles are located at discrete locations in a fluid such as a fluid stream in a flow cytometer. Exemplary formats for distinguishing probe-fragment hybrids for detection of individual typable loci are set forth in further detail below.

Particles useful in the invention are often referred to as microspheres or beads. However, such particles need not be spherical. Rather particles having other shapes including, but not limited to, disks, plates, chips, slivers or irregular shapes can be used. In addition, particles used in the invention can be porous, thus increasing the surface area available for attachment or assay of probe-fragment hybrids. Particle sizes can range, for example, from a few nanometers to many millimeters in diameter as desired for a particular application. For example, particles can be at least about 0.1 micron, 0.5 micron, 1 micron, 10 micron or 100 microns or larger in average diameter. The composition of the beads can vary depending, for example, on the application of the invention or the method of synthesis. Suitable bead compositions include, but are not limited to, those used in peptide, nucleic acid and organic moiety synthesis, such as plastics, ceramics, glass, polystyrene, methylstyrene, acrylic polymers, paramagnetic materials, thoria sol, carbon graphite, titanium dioxide, latex or cross-linked dextrans such as Sepharose™, cellulose, nylon, cross-linked micelles or Teflon™. Useful particles are described, for example, in Microsphere Detection Guide from Bangs Laboratories, Fishers Ind.

Several embodiments of array-based detection in the invention are exemplified below for beads or microspheres. Those skilled in the art will recognize that particles of other shapes and sizes, such as those set forth above, can be used in place of beads or microspheres exemplified for these embodiments.

In some embodiments, polymer probes such as nucleic acids or peptides can be synthesized by sequential addition of monomer units directly on a solid support such as a bead or slide surface. Methods known in the art for synthesis of a variety of different chemical compounds on solid supports can be used in the invention, such as methods for solid phase synthesis of peptides, organic moieties, and nucleic acids. Alternatively, probes can be synthesized first, and then covalently attached to a solid support. Probes can be attached to functional groups on a solid support. Functionalized solid supports can be produced by methods known in the art and, if desired, obtained from any of several commercial suppliers for beads and other supports having surface chemistries that facilitate the attachment of a desired functionality by a user. Exemplary surface chemistries that are useful in the invention include, but are not limited to, amino groups such as aliphatic and aromatic amines, carboxylic acids, aldehydes, amides, chloromethyl groups, hydrazide, hydroxyl groups, sulfonates or sulfates. If desired, a probe can be attached to a solid support via a chemical linker. Such a linker can have characteristics that provide, for example, stable attachment, reversible attachment, sufficient flexibility to allow desired interaction with a genome fragment having a typable locus to be detected, or to avoid undesirable binding reactions. Further exemplary methods that can be used in the invention to attach polymer probes to a solid support are described in Pease et al., Proc. Natl. Acad. Sci. USA 91(11):5022-5026 (1994); Khrapko et al., Mol Biol (Mosk) (USSR) 25:718-730 (1991); Stimpson et al., Proc. Natl. Acad. Sci. USA 92:6379-6383 (1995) or Guo et al., Nucleic Acids Res. 22:5456-5465 (1994).

In embodiments including bead-based arrays, the arrays can be made, for example, by adding a solution or slurry of the beads to a substrate containing attachment sites for the beads. A carrier solution for the beads can be a pH buffer, aqueous solvent, organic solvent, or mixture. Following exposure of a bead slurry to a substrate, the solvent can be evaporated, and excess beads removed. Beads can be loaded into the wells of a substrate, for example, by applying energy such as pressure, agitation or vibration, to the beads in the presence of the wells. Methods for loading beads onto array substrates that can be used in the invention are described, for example, in U.S. Pat. No. 6,355,431.

Probes or particles having attached probes can be randomly deposited on a substrate and their positions in the resulting array determined by a decoding step. This can be done before, during or after the use of the array to detect concatenated nucleic acid targets or other nucleic acids. In embodiments where the placement of probes is random, an encoding or decoding system can be used to localize and/or identify the probes at each location in the array. This can be done in any of a variety of ways, as is described, for example, in U.S. Pat. No. 6,355,431, WO 03/002979 or Gunderson et al., Genome Res. 14:870-877 (2004). As will be appreciated by those in the art, a random array need not necessarily be decoded. For example, beads or probes can be attached to an array substrate, and a detection assay performed to determine the presence of any or all targets independent of identifying which targets are present.

Probes that bind to or are modified in the presence of a nucleic acid target, such as a concatenated nucleic acid target, can be detected in a fluid array. Exemplary formats that can be used in the invention to distinguish beads in a fluid sample using microfluidic devices are described, for example, in U.S. Pat. No. 6,524,793. Commercially available fluid formats for distinguishing beads include, for example, those used in xMAP™ technologies from Luminex or MPSS™ methods from Lynx Therapeutics.

A useful method for making probe arrays is photolithography-based polymer synthesis. For example, Affymetrix® GeneChip® arrays can be synthesized in accordance with techniques sometimes referred to as VLSIPS™ (Very Large Scale Immobilized Polymer Synthesis) technologies. Some aspects of VLSIPS™ and other microarray and polymer (including protein) array manufacturing methods and techniques have been described in U.S. patent application Ser. No. 09/536,841, International Publication No. WO 00/58516; U.S. Pat. Nos. 5,143,854, 5,242,974, 5,252,743, 5,324,633, 5,445,934, 5,744,305, 5,384,261, 5,405,783, 5,424,186, 5,451,683, 5,482,867, 5,491,074, 5,527,681, 5,550,215, 5,571,639, 5,578,832, 5,593,839, 5,599,695, 5,624,711, 5,631,734, 5,795,716, 5,831,070, 5,837,832, 5,856,101, 5,858,659, 5,936,324, 5,968,740, 5,974,164, 5,981,185, 5,981,956, 6,025,601, 6,033,860, 6,040,193, 6,090,555, 6,136,269, 6,269,846, 6,022,963, 6,083,697, 6,291,183, 6,309,831 and 6,428,752; and in PCT Application Nos. PCT/US99/00730 (International Publication No. WO 99/36760) and PCT/US01/04285.

Using VLSIPS™, a GeneChip array can be manufactured by reacting the hydroxylated surface of a quartz wafer with silane. Linkers can then be attached to the silane molecules. The distance between these silane molecules determines the probes' packing density, allowing arrays to hold over 500,000 probe locations, or features, within a mere 1.28 square centimeters. Millions of identical DNA molecules can be synthesized at each feature using a photolithographic process in which masks, carrying 18 to 20 square micron windows that correspond to the dimensions of individual features, are placed over the coated wafer. When ultraviolet light is shone over the mask in the first step of synthesis, the exposed linkers become deprotected and are available for nucleotide coupling. Once the desired features have been activated, a solution containing a single type of deoxynucleotide with a removable protection group can be flushed over the wafer's surface. The nucleotide attaches to the activated linkers, initiating the synthesis process. A capping step can be used to truncate unreacted linkers (or polynucleotides in subsequent step). In the next synthesis step, another mask can be placed over the wafer to allow the next round of deprotection and coupling. The process is repeated until the probes reach their full length, usually 25 nucleotides. However, probes having other lengths such as those set forth elsewhere herein can also be attached at each feature. Once the synthesis is complete, the wafers can be deprotected, diced, and the resulting individual arrays can be packaged in flowcell cartridges.

A spotted array can also be used in a method of the invention. An exemplary spotted array is a CodeLink™ Array available from Amersham Biosciences. CodeLink™ Activated Slides are coated with a long-chain, hydrophilic polymer containing amine-reactive groups. This polymer is covalently crosslinked to itself and to the surface of the slide. Probe attachment can be accomplished through covalent interaction between the amine-modified 5′ end of the oligonucleotide probe and the amine reactive groups present in the polymer. Probes can be attached at discrete locations using spotting pens. Useful pens are stainless steel capillary pens that are individually spring-loaded. Pen load volumes can be less than about 200 nL with a delivery volume of about 0.1 nL or less. Such pens can be used to create features having a spot diameter of, for example, about 140-160 μm. In a preferred embodiment, nucleic acid probes at each spotted feature can be 30 nucleotides long. However, probes having other lengths such as those set forth elsewhere herein can also be attached at each spot.

An array that is useful in the invention can also be manufactured using inkjet printing methods such as SurePrint™ Technology available from Agilent Technologies. Inkjet methods can be used to synthesize oligonucleotide probes in situ or to attach pre-synthesized probes having moieties that are reactive with a substrate surface. A printed microarray can contain 22,575 features on a surface having standard slide dimensions (about 1 inch by 3 inches). Typically, the printed probes are 25 or 60 nucleotides in length. However, probes having other lengths such as those set forth elsewhere herein can also be printed at each location.

An array of arrays or a composite array having a plurality of individual arrays that is configured to allow processing of multiple samples can be used in the invention. Such arrays allow multiplex detection of concatenated nucleic acid targets or other nucleic acids. Exemplary composite arrays that can be used in the invention, for example, in multiplex detection formats include one component systems and two component systems as described in U.S. Pat. No. 6,429,027 and US Pat. App. Pub. No. 2002/0102578. A one component system includes a first substrate having a plurality of assay locations each containing an individual array. For example, one or more wells of a microtiter plate can serve as assay locations and can each contain an array of probes. A two component system includes a first component having an attached array which can be contacted with an assay location, such as a well, of a second component. For example, a first component can include one or more posts each having an array on its end and the first component can be configured such that each array fits within an individual well of a second component such as a microtiter plate. Thus, for some applications the number of individual arrays is set by the number of wells in a microtiter including, for example, 96 well, 384 well and 1536 well microtiter plates corresponding to at most 96, 384 or 1536 individual arrays, respectively. Further exemplary composite arrays that are useful in the invention are described in WO 02/00336, US Pat. App. Pub. No. 02/0102578 or the references cited previously herein in regard to different types of arrays.

Useful substrates for an array or other solid phase support include, but are not limited to, glass; modified glass; functionalized glass; plastics such as acrylics, polystyrene and copolymers of styrene and other materials, polypropylene, polyethylene, polybutylene, polyurethanes, Teflon, or the like; polysaccharides; nylon; nitrocellulose; resins; silica; silica-based materials such as silicon or modified silicon; carbon; metal; inorganic glass; optical fiber bundles, or any of a variety of other polymers. Useful substrates include those that allow optical detection, for example, by being translucent to energy of a desired detection wavelength and/or do not themselves produce appreciable background fluorescence at a particular detection wavelength.

In a particular embodiment, an array substrate can be an optical fiber bundle or array, as is generally described in U.S. Ser. No. 08/944,850, U.S. Pat. No. 6,200,737; WO9840726, and WO9850782. Each optical fiber can have an attached probe or associated particle, the particle being covalently attached to a probe as is generally described in U.S. Pat. Nos. 6,023,540 and 6,327,410. For example, each fiber end can be etched to form a discrete site to which a bead is associated. Similarly other substrates described herein can contain discrete sites for attachment of probes or association of probe bearing particles. The surface of a substrate can be modified to contain discrete sites such as wells, troughs or depressions. Discrete sites can be configured to hold only a single bead or can be configured to hold several beads, as desired. This can be done using a variety of techniques, including, but not limited to, photolithography, stamping techniques, molding techniques or microetching techniques. Those skilled in the art will know or be able to determine an appropriate technique based on the composition and shape of the substrate. The sites on a surface to which particles or probes are attached need not be discrete sites. For example, it is possible to use a uniform surface of adhesive or chemical functionalities that allows the attachment of probes or particles at any position.

A physical barrier, such as a polymer film or membrane can be used over the probes or particles to maintain association with sites and/or protect the probes from degradation. Exemplary polymers include, without limitation, non-naturally occurring polymers such as polyacrylamide, polyvinylpyrrolidine, polymethylacrylate, or polyethylene glycol or derivatives thereof. Naturally occurring polymercan also be used including, for example, hyaluronic acid (poly d-glucuronic acid-n-acetyl-d-glucosamine), cellulose, chitin, starch, gelatin, or agarose. Others include the carrageenans which are a naturally occurring family of polysaccharides derived from red seaweed with names such as Gelcarin, Viscarin and SeaSpen PF (FMC Corp., Philadelphia, Pa.). Other useful stabilization polymers include derivatives of naturally occurring polymers including, for example, Klucel® (hydroxypropylcellulose).

The number of probes in an array can vary depending on the probe composition and desired use of the array. Arrays useful in the invention can have complexity that ranges from about 2 different probes to many millions, billions or higher. The density of an array can be from 2 to as many as a billion or more different probes per square cm. Very high density arrays are useful in the invention including, for example, those having at least about 10,000,000 probes/cm², including, for example, at least about 100,000,000 probes/cm², 1,000,000,000 probes/cm², up to about 2,000,000,000 probes/cm² or higher. High density arrays can also be used including, for example, those in the range from about 100,000 probes/cm² to about 10,000,000 probes/cm². Moderate density arrays useful in the invention can range from about 10,000 probes/cm² to about 100,000 probes/cm². Low density arrays have generally fewer than about 10,000 probes/cm².

Probes, such as those used in an array, can have any of a variety of compositions and characteristics that facilitate their use for detecting a target nucleic acid including, for example, those set forth above in regard to primers and other nucleic acids. Furthermore, molecules other than nucleic acids can be used as probes, so long as they are capable of binding to nucleic acid targets with sufficient specificity for a desired assay set forth herein. Probes can be specific for a particular target sequence that occurs in a gDNA sequence. Alternatively, probes can have sequences that do not substantially complement sequence portions found in a particular gDNA, such as one or more of the genomes described previously herein. Such probes can be included in a universal array that hybridizes to address sequences that have been added exogenously to target nucleic acids, for example, in a probe modification or amplification step, as set forth previously herein.

If desired, nucleic acid probes can be attached to substrates such that they have a free 3′ end for modification by enzymes or other agents. Those skilled in the art will recognize that methods exemplified above in regard to synthesis of nucleic acids in the 3′ to 5′ direction can be modified to produce nucleic acids having free 3′ ends. For example, synthetic methods known in the art for synthesizing nucleic acids in the 5′ to 3′ direction and having 5′ attachments to solid supports can be used in an inkjet printing or photolithographic method. Furthermore, in situ inversion of substrate attached nucleic acids can be carried out such that 3′ substrate-attached nucleic acids become attach to the substrate at their 5′ end and detached at their 3′ end, for example, using methods described in Kwiatkowski et al., Nucl. Acids Res. 27:4710-4714 (1999).

A modified probe, modified target or probe-target complex that contains a label can be distinguished from other molecules that are devoid of the label using methods known in the art. Exemplary properties upon which detection can be based include, but are not limited to, mass, electrical conductivity or optical signals such as a fluorescent signal, absorption signal, luminescent signal, chemiluminescent signal or the like. Detection can also be based on absence or reduced level of one or more signal, for example, due to presence of a signal quenching moiety or degradation of a label moiety.

Detection of fluorescence can be carried out by irradiating a labeled nucleic acid with an excitatory wavelength of radiation and detecting radiation emitted from a fluorophore therein by methods known in the art and described, for example, in Lakowicz, Principles of Fluorescence Spectroscopy, 2nd Ed., Plenum Press New York (1999). A fluorophore can be detected based on any of a variety of fluorescence phenomena including, for example, emission wavelength, excitation wavelength, fluorescence resonance energy transfer (FRET) intensity, quenching, anisotropy or lifetime. FRET can be used to identify hybridization between a first polynucleotide attached to a donor fluorophore and a second polynucleotide attached to an acceptor fluorophore due to transfer of energy from the excited donor to the acceptor. Thus, hybridization can be detected as a shift in wavelength caused by reduction of donor emission and appearance of acceptor emission for the hybrid.

Other detection techniques that can be used to perceive or identify nucleic acids, such as modified probes or concatenated nucleic acid targets, in a method of the invention include, for example, mass spectrometry or electrophoresis which can be used to perceive a nucleic acid based on mass or charge to mass ratio; surface plasmon resonance which can be used to perceive a nucleic acid based on binding to a surface immobilized complementary sequence; absorbance spectroscopy which can be used to perceive a nucleic acid based on the wavelength of absorbed energy; calorimetry which can be used to perceive a nucleic acid based on changes in temperature of its environment upon binding to a complementary sequence; electrical conductance or impedance which can be used to perceive a nucleic acid based on changes in its electrical properties or in the electrical properties of its environment, magnetic resonance which can be used to perceive a nucleic acid based on presence of magnetic nuclei, or other known analytic spectroscopic or chromatographic techniques.

A method of the invention can include a control for monitoring effectiveness of one or more reagent or instrument. For example, in embodiments where methylation of a gDNA sample is identified according to activity of a MSRE, a methylation insensitive isoschizomer of the MSRE can also be reacted with a sample of the gDNA and results for the two enzymes compared. Complete digestions of all sites should occur for the gDNA sample treated with the methylation insensitive isoschizomer, indicating that reagents and instruments used in the assay are functioning correctly. Methylation insensitive isoschizomers are known in the art and include, for example, MspI which recognizes the same sequence as the HpaII MSRE and MboI which recognizes the same sequence as the Sau 3AI MSRE. Other useful isoschizomer pairs are described in the definitions above or in technical materials available from commercial vendors such as New England Biolabs (Beverly, Mass.), Promega (Madison, Wis.), or Invitrogen (Carlsbad, Calif.).

A further control that can be used is a gDNA that has not been treated with an exogenous RE. Such undigested target can serve as a positive control, for example, during a probe hybridization step or a probe modification step. In this regard, the undigested target would be expected to have sequence portions that are not re-ordered and therefore producing a detection signal expected for a concatenated nucleic acid target having sequence portions that remain adjacent due to an intervening inactive RE recognition site.

Another useful calibration control includes artificially methylated gDNA, produced for example, by treatment of gDNA with a site specific methylase. Artificially methylated gDNA can serve as a positive control for identification of in vivo methylated sites in the gDNA. Alternatively, gDNA substantially lacking methylation can be produced by amplifying the gDNA in vitro such that replicated strands, lacking methylation, are present in excess compared to the methylated template. This, non-methylated gDNA can be used as a negative control. Methods for making and using positive and negative methylation controls that can be used in the invention are described, for example, in WO04/05122.

Moreover the level of methylation can be quantitated by calibrating the sample between negative and positive controls. A fully methylation locus should exhibit “full signal”, whereas a monoallelic methylated locus should only exhibit 50% of the signal. Calibration can be performed as described, for example, in WO04/05122.

Typically, amplification conditions used in a method of the invention are chosen to yield minimal amplification bias between uncut loci having inactive RE recognition sites compared to loci having reactive RE recognition sites that are cleaved by the RE. Minimizing bias favors accurate comparison between samples and, therefore, accurate identification of reactive or inactive RE recognition sites. For example, amplification conditions can be chosen to yield minimal amplification bias between loci that are uncut due to presence of a methylated RE recognition site compared to loci that are cleaved due to the presence of an unmethylated RE recognition site.

The methods set forth above have been exemplified with respect to ligating genome fragments to produce a concatenated nucleic acid. However, in particular embodiments, linker adapters can be added to a ligation reaction such that linkers are added to the ends of genome fragments. In such embodiments, attachment of a linker adapter to a genome fragment will indicate that a RE recognition site located at the site of ligation is reactive for restriction by the RE.

A linker adapter can be ligated to a restricted genome fragment using methods known in the art. Furthermore, the ligated product can be amplified, for example, using a primer that binds to the ligated linkers. This method is known in the art as linker adapter PCR. Useful conditions for linker adapter PCR are described, for example, in Kinzler et al., Nucl. Acids Res. 17:3645-3653 (1989); Lucito et al., Genome Res. 10:1726-36 (2000); Matsuzaki et al., Genome Res. 14:414-425 (2004); US Pat. App. Pub. No. 2004/0137473; and U.S. Pat. Nos. 6,361,947 and 6,703,228.

Methods set forth above can be used to detect absence or presence of a linker adjacent to a particular sequence portion. In this regard, the presence of a linker adjacent to a sequence portion can be detected for example due to inhibition of a primer extension event for a probe that hybridizes to a sequence spanning the RE recognition sequence between adjacent portions. Similarly, presence of a linker can be detected due to reduced hybridization of a probe to a portion having a linker adapter compared to the level of hybridization of the probe to a portion having the same contiguity in a concatenated nucleic acid as the gDNA from which fragments were created and concatenated. Furthermore, a positive signal can be detected for a reactive RE recognition site by detecting primer modification due to the presence of the linker adapter sequence adjacent to a portion flanked by the RE recognition site.

The invention further provides a method of identifying a reactivity state of a plurality of recognition sites for a restriction endonuclease in genomic DNA. The method includes the steps of (a) providing an isolated native genomic DNA having a first sequence, wherein the genomic DNA has a plurality of recognition sites for a restriction endonuclease, the recognition sites having recognition sequences in the first sequence, wherein the recognition sites have a first reactivity state or second reactivity state, wherein the genomic DNA has portions of the first sequence that are adjacent to the recognition sequences; (b) cleaving the genomic DNA at the recognition sites with the restriction endonuclease, thereby producing genomic DNA fragments having the portions of the first sequence; (c) ligating the fragments, thereby forming a concatenated DNA having a second sequence, wherein the portions are re-ordered in the second sequence compared to the first sequence if the recognition sites are in the first reactivity state and wherein the portions are ordered the same in the second sequence compared to the first sequence if the recognition sites are in the second reactivity state; and (d) identifying the order for a plurality of the portions, thereby identifying the reactivity state of the plurality of recognition sites for the restriction endonuclease in the genomic DNA.

Also provides is a method of identifying methylation state of a plurality of CpG target sites in genomic DNA. The method includes the steps of (a) providing an isolated native genomic DNA having a first sequence, wherein the genomic DNA has CpG target sites at a plurality of different locations, wherein the CpG target sites have a cytosine having a 5-methyl moiety or a 5-hydrogen moiety, wherein the genomic DNA has portions that are adjacent to the CpG target sites; (b) cleaving the genomic DNA at the CpG target sites that have the 5-methyl cytosine, thereby producing genomic DNA fragments having the portions; (c) ligating the fragments, thereby forming a concatenated DNA having the portions in a second sequence, wherein the portions are re-ordered in the second sequence compared to the first sequence if the CpG target sites have cytosine having the 5-methyl moiety and wherein the portions are ordered the same in the second sequence compared to the first sequence if the CpG target sites have cytosine having the 5-hydrogen moiety; and (d) identifying the order for a plurality of the portions, thereby identifying methylation state of the plurality of CpG target sites in the genomic DNA.

A method of the invention can be used to identify a modification of gDNA, or presence of another factor that influences reactivity of an RE recognition sequence, and to correlate the modification or factor with a particular type of cell or with a particular developmental stage of a cell. In this regard a method of the invention can be carried out to compare two different cells to identify a modification or factor that is specifically associated with one of the cells. Similarly, a modification or factor can be correlated with a particular disease or condition experienced by a cell. Using methods such as those set forth in further detail below, the invention can be used to compare reactivity states for RE recognition sequences for different cells including, for example, cancerous and non-cancerous cells isolated from the same individual or from different individuals.

In particular embodiments, the invention can be used to diagnose an individual with a condition that is characterized by a level and/or pattern of methylated genomic CpG dinucleotide sequences distinct from the level and/or pattern of methylated genomic CpG dinucleotide sequences exhibited in the absence of the condition. The methods can also be used to predict the susceptibility of an individual to a condition that is characterized by a level and/or pattern of methylated genomic CpG dinucleotide sequences that is distinct from the level and/or pattern of methylated genomic CpG dinucleotide sequences exhibited in the absence of the condition.

A method of the invention can be employed to detect altered levels of methylation of genomic CpG dinucleotide sequences in a biological sample compared to a reference level. Furthermore, the methods can be used to determine methylation patterns, which are represented by differential methylation of selected genomic CpG dinucleotide sequences that serve as markers in particular sets or subsets of genomic targets. In embodiments directed to the detection of methylation patterns, it is possible to diagnose or predict the susceptibility of an individual to a specific tumor-type based on the correlation between the pattern and the tumor type.

The level of methylation of differentially methylated genomic CpG dinucleotide sequences can provide a variety of information about a disease or condition and can be used, for example, to diagnose cancer in the individual; to predict the course of the cancer in the individual; to predict the susceptibility to cancer in the individual, to stage the progression of the cancer in the individual; to predict the likelihood of overall survival for the individual; to predict the likelihood of recurrence of cancer for the individual; or to determine the effectiveness of a treatment course undergone by the individual.

A level of methylation that is detected in a biological sample can be decreased or increased in comparison to a reference level and alterations that increase or decrease methylation can be detected and provide useful prognostic or diagnostic information. For example, hypermethylation of CpG islands located in the promoter regions of tumor suppressor genes have been established as common mechanisms for gene inactivation in cancers (Esteller, Oncogene 21(35): 5427-40 (2002)). Thus, a detailed study of methylation pattern in selected, staged tumor samples compared to matched normal tissues from the same patient can identify unique molecular markers for cancer classification.

In addition to detecting levels of methylation, the present invention also allows for the detection of patterns of methylation. Analysis of methylation patterns across one or more chromosome in biological samples from afflicted individuals can reveal epigenetic changes in the form of altered levels of methylation of subsets of genomic CpG dinucleotide sequences that make up a pattern of affected genomic targets that can be correlated with a condition.

A method of the invention can be used for prognosis, for example, to identify surgically treated patients likely to experience recurrence of a condition or disease such as cancer so that they can be offered additional therapeutic options, including preoperative or postoperative adjuncts such as chemotherapy, radiation, biological modifiers and other suitable therapies. Thus, the methods can be used for determining the risk of metastasis in patients who demonstrate no measurable metastasis at the time of examination or surgery.

A prognostic method of the invention can also be useful for determining a proper course of treatment for a patient having a condition or disease such as cancer. A course of treatment refers to the therapeutic measures taken for a patient after diagnosis or after treatment. For example, a determination of the likelihood for cancer recurrence, spread, or patient survival, can assist in determining whether a more conservative or more aggressive approach to therapy should be taken, or whether treatment modalities should be combined. For example, when cancer recurrence is likely, it can be advantageous to precede or follow surgical treatment with chemotherapy, radiation, immunotherapy, biological modifier therapy, gene therapy, vaccines, and the like, or to adjust the span of time during which the patient is treated.

In accordance with another embodiment of the present invention, there are provided diagnostic systems for carrying out one or more of the methods described previously herein. A diagnostic system of the invention can be provided in kit form including, if desired, a suitable packaging material. In one embodiment, for example, a diagnostic system can include a plurality of nucleic acid probes, for example, in an array format, and one or more reagents useful for detecting a concatenated nucleic acid target or other target nucleic acid hybridized to a probe of the array. Accordingly, any combination of reagents or components that is useful in a method of the invention, such as those set forth herein previously in regard to particular methods, can be included in a kit provided by the invention. For example, a kit can include one or more nucleic acid probes bound to an array and having free 3′ ends along with other reagents useful for a primer extension detection reaction.

A packaging material can include one or more physical structures used to house the contents of a kit, such as nucleic acid probes or primers, or the like. The packaging material can be constructed by well known methods, preferably to provide a sterile, contaminant-free environment. The packaging materials employed herein can include, for example, those customarily utilized in nucleic acid-based diagnostic systems. Exemplary packaging materials include, without limitation, glass, plastic, paper, foil, and the like, capable of holding within fixed limits a component useful in the methods of the invention such as an isolated nucleic acid, oligonucleotide, or primer.

The packaging material can include a label which indicates that the invention nucleic acids can be used for a particular method. For example, a label can indicate that the kit is useful for detecting a particular RE recognition site or a particular modification of a site, such as methylation.

Instructions for use of the packaged reagents or components are also typically included in a kit of the invention. The instructions for use typically include a tangible expression describing the reagent or component concentration or at least one assay method parameter, such as the relative amounts of kit components and sample to be admixed, maintenance time periods for reagent/sample admixtures, temperature, buffer conditions, and the like.

Throughout this application various publications, patents or patent applications have been referenced. The disclosure of these publications in their entireties are hereby incorporated by reference in this application in order to more fully describe the state of the art to which this invention pertains.

The term “comprising” is intended herein to be open-ended, including not only the recited elements, but further encompassing any additional elements.

Although the invention has been described with reference to the examples provided above, it should be understood that various modifications can be made without departing from the invention. Accordingly, the invention is limited only by the claims.

What is claimed is:

1. A method of identifying a plurality of reactive recognition sites for a restriction endonuclease in genomic DNA, comprising
(a) providing an isolated native genomic DNA comprising a first sequence,
wherein said genomic DNA comprises a plurality of reactive recognition sites for a restriction endonuclease;
(b) cleaving said genomic DNA at said recognition sites with said restriction endonuclease, thereby producing genomic DNA fragments comprising portions of said first sequence;
(c) ligating said fragments, thereby forming a concatenated DNA comprising said portions in a second sequence, wherein said portions are re-ordered in said second sequence compared to said first sequence; and
(d) identifying a plurality of said portions that are re-ordered, thereby identifying a plurality of reactive recognition sites for said restriction endonuclease in said genomic DNA.

2. The method of claim 1, wherein said plurality of recognition sites comprises at least 10,000 sites.

3. The method of claim 1, wherein said first sequence comprises at least 10 kb.

4. The method of claim 1, wherein said genomic DNA is isolated from a mammal.

5. The method of claim 1, wherein said restriction endonuclease is methylation sensitive and said reactive restriction sites are unmethylated, thereby identifying a plurality of unmethylated recognition sites for said restriction endonuclease in said genomic DNA.

6. The method of claim 1, wherein step (d) comprises identifying a plurality of said portions that are re-ordered, thereby identifying at least 10 reactive recognition sites for a restriction endonuclease in said genomic DNA.

7. The method of claim 1, further comprising amplifying said concatenated DNA, thereby producing a nucleic acid target comprising at least one of said portions.

8. The method of claim 7, wherein said amplifying comprises random primer amplification using said concatenated DNA as template.

9. The method of claim 7, further comprising fragmenting said nucleic acid target.

10. The method of claim 7, wherein said nucleic acid target is produced under isothermal conditions.

11. The method of claim 1, wherein said identifying comprises hybridizing a probe to a nucleic acid target comprising at least one of said portions.

12. The method of claim 11, wherein said target nucleic acid comprises said concatenated DNA.

13. The method of claim 11, wherein said target nucleic acid comprises a product of amplification of said concatenated DNA.

14. The method of claim 11, further comprising modifying said probe while bound to said nucleic acid target.

15. The method of claim 14, wherein said modifying comprises polymerase catalyzed addition of a nucleotide or nucleotide analog to said probe.

16. The method of claim 1, wherein said identifying comprises hybridizing an array of nucleic acid probes to a plurality of nucleic acid targets each comprising at least one of said portions.

17. The method of claim 1, further comprising fragmenting said concatenated DNA.

18. A method of identifying a reactivity state of a plurality of recognition sites for a restriction endonuclease in genomic DNA, comprising
(a) providing an isolated native genomic DNA comprising a first sequence,
wherein said genomic DNA comprises a plurality of recognition sites for a restriction endonuclease, said recognition sites having recognition sequences in said first sequence,
wherein said recognition sites comprise a first reactivity state or second reactivity state,
wherein said genomic DNA comprises portions of said first sequence that are adjacent to said recognition sequences;
(b) cleaving said genomic DNA at said recognition sites with said restriction endonuclease, thereby producing genomic DNA fragments comprising said portions of said first sequence;
(c) ligating said fragments, thereby forming a concatenated DNA comprising a second sequence, wherein said portions are re-ordered in said second sequence compared to said first sequence if said recognition sites are in said first reactivity state and wherein said portions are ordered the same in said second sequence compared to said first sequence if said recognition sites are in said second reactivity state; and
(d) identifying the order for a plurality of said portions, thereby identifying said reactivity state of said plurality of recognition sites for said restriction endonuclease in said genomic DNA.

19. A method of identifying methylation state of a plurality of CpG target sites in genomic DNA, comprising
(a) providing an isolated native genomic DNA comprising a first sequence,
wherein said genomic DNA comprises CpG target sites at a plurality of different locations,
wherein said CpG target sites comprise a cytosine having a 5-methyl moiety or a 5-hydrogen moiety,
wherein said genomic DNA comprises portions that are adjacent to said CpG target sites;
(b) cleaving said genomic DNA at said CpG target sites that comprise said 5-methyl cytosine, thereby producing genomic DNA fragments comprising said portions;
(c) ligating said fragments, thereby forming a concatenated DNA comprising said portions in a second sequence, wherein said portions are re-ordered in said second sequence compared to said first sequence if said CpG target sites comprise cytosine having said 5-methyl moiety and wherein said portions are ordered the same in said second sequence compared to said first sequence if said CpG target sites comprise cytosine having said 5-hydrogen moiety; and
(d) identifying the order for a plurality of said portions, thereby identifying methylation state of said plurality of CpG target sites in said genomic DNA.
